(19) United States
(12) Patent Application Publication
(10) Pub. No.: US 2004/0142894 A1
(43) Pub. Date: Jul. 22, 2004

(54) Modulation of cellular proliferation

(76) Inventors: Gary S. Stein, Shrewsbury, MA (US); Andre J. Van Wijnen, Auburn, MA (US); Janet L. Stein, Shrewsbury, MA (US); Partha Mitra, Worcester, MA (US); Ronglin Xie, Worcester, MA (US)

Correspondence Address: FISH & RICHARDSON PC, 225 FRANKLIN ST, BOSTON, MA, 02110, US

(21) Appl. No.: 10/694,711

(22) Filed: Oct. 27, 2003

Related U.S. Application Data
(60) Provisional application No. 60/421,166, filed on Oct. 25, 2002.

(51) Int. Cl. A61K 48/00; C12N 15/85

(52) U.S. Cl. 514/44; 435/455

(57) ABSTRACT

The invention relates to methods and compositions for modulating cellular proliferation using HiNF-P and NPAT. Also included are methods of identifying compounds that modulate cell proliferation by modulating HiNF-P expression and activity.

### CLAIM OF PRIORITY

This application claims priority under 35 USC §119(e) to U.S. Patent Application Serial No. 60/421,166, filed on Oct. 25, 2002, the entire contents of which are hereby incorporated by reference.

### TECHNICAL FIELD

This invention relates to regulation of the eukaryotic cell cycle, more specifically to regulation of histone H4 expression.

### BACKGROUND

The G1/S phase transition is a critical stage during the somatic cell cycle that defines cellular commitment to replicate the genome and progress towards mitotic division. Passage beyond the G1/S boundary depends initially on the activation of the cyclin/cyclin dependent kinase (CDK) cascade by growth factors' and the induction of the CDK2/cyclin E kinase complex at the restriction (R) point. At the onset of S phase, de novo synthesis of histone proteins is required to package nascent DNA into chromatin immediately upon initiation of DNA synthesis. The stringent coupling between histone biosynthesis and DNA replication necessitates the coordinate transcriptional activation of the fourteen distinct human genes encoding histone H4, a highly conserved nucleosomal protein.

Histone H4 gene transcription is regulated by the multiple sequence-specific transcription factors, Histone Nuclear Factor P (HiNF-P), Histone Nuclear Factor D (HiNF-D), and Histone Nuclear Factor M (HiNF-M), which bind to the principal regulatory domain, Site II, that resides in the proximal promoter of histone H4 genes. Site II mediates cell cycle control of transcription at the onset of S phase and is phylogenetically conserved among multiple histone H4 genes in metazoan species.

NPAT (nuclear protein mapped to the ATM locus) is a direct downstream target of the cyclin E/CDK2 signaling pathway. ATM is a locus that is associated with ataxia-telangiectasia (A-T), an autosomal recessive disorder involving cerebellar degeneration, immunodeficiency, chromosomal instability, radiosensitivity, and a predisposition to cancer. NPAT is essential for normal mammalian development and enhances histone gene transcription. However, NPAT does not bind directly to DNA, and the mediator that transduces the NPAT/cyclin E/CDK2 signal at the Site II cell cycle regulatory element of the histone H4 gene promoter has yet to be identified.

### SUMMARY

The invention is based, in part, on the discovery that HiNF-P (Histone Nuclear Factor P) enhances H4 histone gene transcription, which in turn activates the cell cycle phase transition of G1 to S, resulting in cell proliferation. Thus, increasing the expression of HiNF-P in a cell can induce or enhance cell proliferation. It has also been discovered that the increased expression of HiNF-P and NPAT (nuclear protein mapped to the ATM locus) enhances H4 histone gene transcription above the levels measured by the addition of either protein individually, and cdk2/cyclin E phosphorylation sites on HiNF-P are required for activity. Furthermore, it has been discovered that HiNF-P and NPAT physically associate with each other, thus, HiNF-P is the final link that couples growth factor stimulation and the concomitant activation of the cdk2/cyclin E/NPAT signaling cascade with induction of histone H4 gene transcription at the G1/S phase transition. Finally, HiNF-P has been discovered to be a bifunctional regulator, with inhibitory or activating activity on H4 histone gene transcription, depending on the kinase status of the cdk2/cyclin E/NPAT signaling pathway. Compounds that block the activity of cdk2/cyclin E activity therefore reduce the activity of HiNF-P at the Site II promoter.

In addition, compounds that inhibit the expression or activity of HiNF-P inhibit H4 histone gene transcription, thereby inhibiting cell proliferation; conversely, compounds that enhance the expression or activity of HiNF-P activate H4 histone gene transcription and increase cell proliferation.

The invention includes compounds and methods that can be used to alter HiNF-P activity and thus manipulate the proliferative state of a cell. Therefore, cell proliferation can be therapeutically regulated, e.g., inhibited (e.g., to inhibit proliferation of cancerous cells) or enhanced (e.g., for wound healing, to increase growth of cells such as blood, hair, or skin cells, increase angiogenesis, or for tissue or organ regeneration or rebuilding) with the compounds and methods described herein. Additionally, the compounds and methods described herein can be used to promote the growth of progenitors along different cell lineages, or to modulate the ex vivo expansion of cells, e.g., for cell transplantation or gene therapy.

Accordingly, the invention includes methods of enhancing cellular proliferation by increasing HiNF-P activity or expression. In some embodiments, the methods include introducing into a cell a nucleic acid molecule encoding a HiNF-P polypeptide and culturing the cell under conditions such that the nucleic acid molecule encoding the HiNF-P polypeptide is expressed. The nucleic acid molecule encoding the HiNF-P polypeptide can be in an expression vector. In some embodiments, the methods include introducing into the cell a nucleic acid sequence encoding an NPAT polypeptide and culturing the cell under conditions such the nucleic acid molecule encoding the NPAT polypeptide is expressed. The nucleic acid molecule encoding the NPAT polypeptide can be in an expression vector.

In some embodiments, the method includes a method of enhancing cellular proliferation, by introducing into a cell a compound that alters the expression or activity of a Histone Nuclear Factor P (HiNF-P) polypeptide, in an amount effective to enhance proliferation of the cell. The method of claim 1, wherein the compound is a nucleic acid molecule comprising a sequence encoding a HiNF-P polypeptide, which is introduced into the cell under conditions such that the HiNF-P polypeptide is expressed. In some embodiments, the nucleic acid molecule encoding the HiNF-P polypeptide is in an expression vector. In some embodiments, the compound is an HiNF-P polypeptide. In some embodiments, the compound increases the number of HiNF-P polypeptides. In some embodiments, the compound increases a biological activity of the HiNF-P polypeptide. In some embodiments, the biological activity that is increased results in enhanced expression of a histone H4 gene. In some embodiments, the cell is in a living mammal, e.g., a human or other non-human mammal. In some embodiments, the method also includes introducing into the cell a nucleic acid molecule encoding an Nuclear Protein, Ataxia-Telangiectasia locus (NPAT) polypeptide, under conditions such the NPAT polypeptide is expressed. In some embodiments, the nucleic acid molecule encoding the NPAT polypeptide is in an expression vector.

In some embodiments, the mammal has a condition that would benefit from increased cell proliferation, e.g., a wound, and the compound enhances healing of the wound. In some embodiments, the cell is in or near the wound. A cell that is “near” the wound is sufficiently close to the wound that a compound, e.g., a HiNF-P polypeptide, can traverse the intervening distance by diffusion. Thus the invention includes methods of treating a subject having a condition that would benefit from increased cell proliferation, e.g., for wound healing, angiogenesis, or tissue or organ growth or regeneration, by administering to the subject an effective amount of a compound that increases HiNF-P expression or activity. In some embodiments, the wound is in the skin, and the compound is administered topically.

In some embodiments, the cell is a cultured cell. Thus, in other embodiments, the methods of enhancing cellular proliferation include contacting a cell with a compound that increases expression or activity of a HiNF-P polypeptide and culturing the cell under conditions such that the HiNF-P polypeptide is expressed. The cell can contain a nucleic acid molecule encoding a HiNF-P polypeptide in an expression vector.

The invention also includes methods for expressing a target nucleic acid sequence (e.g., a reporter gene) in a cell. The methods include introducing an expression vector into a cell (the vector contains a HiNF-P binding sequence (e.g., a Site II promoter sequence) operatively linked to the target sequence and regulatory elements effective to enable transcription of the target nucleic acid sequence in the cell when HiNF-P polypeptide is expressed in the cell), and culturing the cell or progeny of the cell under conditions that enable expression of HiNF-P polypeptide, thus increasing transcription of the target nucleic acid sequence. The vector can contain at least two copies of the HiNF-P binding site. In some embodiments, the transcription nucleic acid sequence is also translated. In some embodiments, an expression vector including a nucleic acid sequence encoding a HiNF-P polypeptide is introduced into the cell and HiNF-P polypeptide is expressed in the cell. In another embodiment, an expression vector containing a nucleic acid sequence encoding an NPAT polypeptide is also introduced into the cell, and NPAT polypeptide is expressed in the cell. In other embodiments, a candidate modulator of HiNF-P activity or expression is introduced into the cell, and the levels of expression of the target nucleic acid are determined, e.g., in the presence and in the absence of the candidate modulator.

Thus, in another aspect, the invention includes a method of increasing transcription of a pre-selected target nucleic acid sequence in a cell. The method includes introducing an expression vector into a cell, e.g., a vector including a Histone Nuclear Factor P (HiNF-P) binding site (e.g., a Site II promoter sequence) operatively linked to the target sequence, and a regulatory element effective to enable transcription of the target nucleic acid sequence in the cell when a HiNF-P polypeptide is expressed in the cell; and culturing the cell or progeny of the cell under conditions that enable expression of the HiNF-P polypeptide. In some embodiments, the vector includes at least two copies of the HiNF-P binding site. In some embodiments, the transcribed nucleic acid sequence is translated. In some embodiments, the method includes introducing into the cell an expression vector including a nucleic acid sequence encoding a HiNF-P polypeptide, and culturing the cell to express the HiNF-P polypeptide. In some embodiments, the method also includes introducing into the cell an expression vector including a nucleic acid sequence encoding a Nuclear Protein, Ataxia-Telangiectasia locus (NPAT) polypeptide, and culturing the cell to express the NPAT polypeptide.

In another aspect, the invention includes an isolated polypeptide consisting of amino acids 394 to 411 of SEQ ID NO:6 (VRYESVELTQ QLLRQPQE; SEQ ID NO:3). Also included is an isolated polypeptide consisting of amino acids 502 to 517 of SEQ ID NO:6 (MEKLQGIAEE PEIQMV; SEQ ID NO: 4).

In another aspect, the invention includes an isolated antibody or portion thereof that specifically binds to a HiNF-P polypeptide. In some cases, the isolated antibody specifically binds to a region within amino acids 394 to 411 of SEQ ID NO:6 (VRYESVELTQ QLLRQPQE; SEQ ID NO:3). In another aspect, the isolated antibody specifically binds to a region within amino acids 502 to 517 of SEQ ID NO:6 (MEKLQGIAEE PEIQMV; SEQ ID NO: 4).

In another aspect, the invention relates to a method of inhibiting cell proliferation by inhibiting HiNF-P expression or activity. The method includes obtaining a cell and contacting the cell with an agent that can inhibit HiNF-P expression or activity in the cell. In some cases, HiNF-P expression is inhibited by introducing into the cell an agent, e.g., a nucleic acid, e.g., a HiNF-P antisense oligonucleotide or an siRNA that is targeted to a HiNF-P nucleic acid sequence. In some embodiments, NPAT expression or activity is inhibited. In other embodiments, the activity of the cdk2/cyclin E kinase is inhibited, e.g., by administering an inhibitor of the kinase, e.g., p57.

In another aspect, the invention relates to a method of identifying a compound that modulates expression of an H4 histone gene. The method includes obtaining a cell comprising:

(a) a reporter gene operatively linked to a Histone Nuclear Factor P (HiNF-P) binding site (e.g., a Site II promoter sequence), such that when a HiNF-P polypeptide binds to the HiNF-P binding site the reporter gene is expressed, and

(b) a nucleic acid sequence encoding a HiNF-P polypeptide that is expressed. The cell is contacted with a test compound; and the level of expression of the reporter gene is measured. A difference in the level of expression of the reporter gene (e.g., an increase or decrease in the level of expression) relative to a control cell that was not contacted with the test compound indicates that the test compound modulates the expression of a H4 histone gene. In certain embodiments, the reporter gene is an H4 histone gene, which is naturally linked to a HiNF-P binding sequence, e.g., an endogenous H4 histone gene and Site II sequence, and the H4 histone gene is expressed when the HiNF-P polypeptide binds to the Site II sequence.

In some embodiments, the test compound is an antisense oligonucleotide, an siRNA, a peptidomimetic, peptoid, aptamer, carbohydrate, polysaccharide, non-nucleic acid small organic molecule, inorganic molecule, polypeptide, antibody, or ribozyme.

In some embodiments, the invention relates to an isolated antisense nucleotide sequence that inhibits expression of a HiNF-P polypeptide, e.g., 5′-GGCATTGGTCTGATTCACC-3′ (SEQ ID NO:10).

In another aspect, the invention relates to a method of decreasing proliferation of a cell. The method includes administering to the cell a composition in an amount sufficient to inhibit Histone Nuclear Factor P (HiNF-P) expression or activity in the cell, thereby decreasing proliferation of the cell. In some embodiments, the compound administered to the cell is a HiNF-P antisense oligonucleotide or an siRNA that is targeted to a HiNF-P nucleic acid sequence. In some embodiments, the method also includes administering a second composition in an amount sufficient to inhibit Nuclear Protein, Ataxia-Telangiectasia locus (NPAT) expression or activity. In some embodiments, the method is used to treat a disorder associated with excessive proliferation, e.g., cancer.

The invention also includes a method for detecting a HiNF-P polypeptide in a biological sample. The method includes the steps of obtaining a biological sample, contacting the biological sample with an antibody that specifically binds to a HiNF-P polypeptide under conditions that allow the formation of a HiNF-P polypeptide-antibody complex, and detecting the HiNF-P polypeptide-antibody complex, such that the presence of a complex indicates the presence of a HiNF-P polypeptide in the sample.

In another embodiment, the invention relates to a method of diagnosing a disorder (e.g., cancer) associated with aberrant expression of a HiNF-P polypeptide. The method includes the steps of measuring HiNF-P polypeptide expression in a tissue sample from a subject suspected of having a disorder associated with aberrant HiNF-P expression, such that increased or decreased HiNF-P polypeptide expression in the tissue sample compared to a control indicates that the subject has a disorder associated with aberrant expression of a HiNF-P polypeptide. In some embodiments, the disorder is cancer.

The invention also relates to methods of treating a subject having a disorder characterized by aberrant (e.g., increased, excessive, or rapid) cell proliferation (e.g., cancer or psoriasis.

Thus the invention includes a method of treating a subject having a disorder characterized by excessive cell proliferation (e.g., cancer or psoriasis), by administering to the subject a compound that inhibits Histone Nuclear Factor P (HiNF-P) expression or activity, in an amount effective to decrease cell proliferation. In some embodiments, the compound administered to the subject is a HiNF-P antisense oligonucleotide or an siRNA that is targeted to a HiNF-P nucleic acid sequence.

In another aspect, the invention relates to a transgenic non-human mammal whose somatic and germ cells comprise a disrupted Histone Nuclear Factor P (HiNF-P) allele, wherein the cells, if the mouse is homozygous, exhibit decreased HiNF-P activity as compared to a wildtype mouse. In some embodiments, the mammal is a mouse. In some embodiments, the cells of the mammal comprise two disrupted HiNF-P alleles. In some embodiments, the cells of the mammal additionally comprise a disrupted Nuclear Protein, Ataxia-Telangiectasia locus (NPAT) allele.

In some embodiments, the cells of the mammal comprise a conditional transgene that modulates the expression of a Histone Nuclear Factor P (HiNF-P) polypeptide, wherein under pre-selected conditions the transgene increases or decreases the expression of the HiNF-P polypeptide. In some embodiments, the conditional transgene decreases the expression of the HiNF-P polypeptide. In some embodiments, the conditional transgene increases the expression of the HiNF-P polypeptide.

In another aspect, the invention relates to a targeting vector comprising a nucleic acid sequence that disrupts expression of a Histone Nuclear Factor P (HiNF-P) polypeptide when introduced into a cell, the vector comprising a first and second sequences complementary to first and second regions of a nucleic acid sequence encoding a HiNF-P polypeptide, and a third sequence inserted between the first and second sequences, wherein the third sequence is not complementary to a nucleic acid sequence encoding a HiNF-P polypeptide. In some embodiments, the third sequence is a reporter gene, e.g., a gene that encodes β-galactosidase. In some embodiments, the nucleic acid sequence disrupts expression of a HiNF-P polypeptide by homologous recombination into an endogenous HiNF-P allele. In some embodiments, the targeting vector of claim also includes a regulatory sequence capable of conditionally disrupting expression of a HiNF-P allele, e.g., utilizes a Lox sequence (Cre-Lox technology).

In some aspects, the invention relates to a therapeutic composition comprising a compound that modulates the expression or activity of a Histone Nuclear Factor P (HiNF-P) polypeptide, and a pharmaceutically acceptable carrier. In some embodiments, the compound is a Histone Nuclear Factor P (HiNF-P) polypeptide or biologically active fragment thereof, an anti-HiNF-P antibody, antisense, or siRNA, or active fragment of NPAT.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded.

An “isolated nucleic acid” or a “purified nucleic acid molecule” refers to a nucleic acid molecule that is separated from other nucleic acid molecules that are present in the natural source of the nucleic acid. For example, with reference to genomic DNA, the term “isolated nucleic acid” includes a nucleic acid molecule that is separated from the chromosome with which the genomic DNA of interest is naturally associated. In general, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. Moreover, an “isolated” nucleic acid molecule, such as a cDNA molecule, is typically substantially free of other cellular material, or culture medium when produced by recombinant techniques, or substantially free of chemical precursors or other chemicals when chemically synthesized.

A nucleic acid molecule that “hybridizes under stringent conditions” hybridizes under stringent conditions that are known to those skilled in the art, such as those in Current Protocols in Molecular Biology, John Wiley & Sons, N.Y. (1989), 6.3.1-6.3.6. Aqueous and nonaqueous methods are described in that reference and either can be used. Examples of hybridization conditions are hybridization in 6× sodium chloride/sodium citrate (SSC) at about 45° C., followed by one or more washes in 0.2×SSC, 0.1% SDS at 50° C. (low stringency), 55° C. (moderate stringency), or 60° C. (high stringency), depending on the level of stringency required.

A “naturally-occurring” nucleic acid molecule is an RNA or DNA molecule having a nucleotide sequence that occurs in nature (e.g., encodes a natural protein).

A “polypeptide” is any chain of amino acids, regardless of length, and thus includes polypeptides, proteins, and peptides.

By “substantially pure” is meant a preparation that is at least 60% by weight (dry weight) the compound of interest, i.e., a HiNF-P polypeptide. A preparation of a substantially pure HiNF-P polypeptide is also substantially free of an antibody or any other compound that binds to a HiNF-P polypeptide.

In the case of polypeptide sequences that are less than 100% identical to a reference sequence, e.g., in a mutant polypeptide or an analog of a wild-type protein, the non-identical positions can be conservative substitutions for the reference sequence or substitutions of non-essential amino acids. A “non-essential” amino acid residue is a residue that can be altered from the wild-type amino acid sequence of a HiNF-P (e.g., the sequence of SEQ ID NO: 6) without substantially altering a biological activity of the polypeptide, whereas an “essential” amino acid residue results in such a change. The biological activities of a HiNF-P include binding to Site II nucleic acid sequence, and the ability to enhance histone H4 expression.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a HiNF-P polypeptide is generally replaced with another amino acid residue from the same side chain family. Alternatively, in another embodiment, mutations can be introduced randomly along all or part of a HiNF-P coding sequence, such as by saturation mutagenesis, and the resultant mutants can be screened for HiNF-P biological activity to identify mutants that retain activity. Following mutagenesis, the nucleotide sequence of the HiNF-P nucleic acid sequence can be expressed recombinantly and the activity of the protein can be determined. Such mutated HiNF-P sequences are useful, e.g., for modulating histone H4 expression.

As used herein, a “biologically active portion” of a HiNF-P polypeptide includes a fragment of a HiNF-P polypeptide that participates in an interaction between a HiNF-P molecule and a non-HiNF-P molecule (e.g., NPAT and/or the Site II promoter sequence). Biologically active portions of a HiNF-P polypeptide include peptides having an amino acid sequence that is identical to, sufficiently homologous to, or derived from the amino acid sequence of the HiNF-P polypeptide, e.g., the amino acid sequence that is shown in SEQ ID NO:6, have fewer amino acids than the full length HiNF-P polypeptides, and exhibit at least one activity of a HiNF-P polypeptide. Typically, biologically active portions include a domain or motif with at least one activity of the HiNF-P polypeptide, e.g., the ability to associate with NPAT and/or the ability to associate with Site II promoter sequence and/or the ability to activate or inhibit a histone H4 gene. A biologically active portion of a HiNF-P polypeptide can be a polypeptide, which is, for example, 10, 25, 50, 100, 200 or more amino acids in length. Biologically active portions of a HiNF-P polypeptide can be used, e.g., as agents that modulate a HiNF-P mediated activity, or as targets for developing such agents.

An antibody that “specifically binds” to an antigen is an antibody that binds to a particular antigen, e.g., a HiNF-P polypeptide, but that does not significantly bind to other molecules or proteins in a sample, e.g., a biological sample, that contains a HiNF-P polypeptide.

“Aberrant expression,” or “mis-expression,” as used herein, refers to a non-wildtype pattern of gene expression, at the RNA or protein level. It includes: expression at non-wildtype levels, i.e., over or under expression; a pattern of expression that differs from wild type in terms of the time or stage at which a gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

A “subject” can be a human or an animal, e.g., a mammal, such as a horse, cow, goat, sheep, or other domestic mammal, or a mouse or rat. This includes animals that are used as models of disease, in particular models for proliferative disorders such as cancer. Subjects can also be non-mammalian animals, such as birds, e.g., domestic poultry, fish (e.g., zebrafish), reptiles, amphibians, and invertebrates such as Drosophila melanogaster and Caenorhabditis elegans.

A “purified preparation of cells” refers to, in the case of plant or animal cells, an in vitro preparation of cells and not an entire intact plant or animal. In the case of cultured cells or microbial cells, it consists of a preparation wherein, of the total number of cells in the preparation, at least 50% are the desired cells.

A “transformed cell” is a cell into which (or into an ancestor of which) has been introduced, by means of recombinant DNA techniques, a nucleic acid molecule or transgene encoding a HiNF-P polypeptide.

As used herein, the term “operatively linked” means that a selected nucleic acid sequence, e.g., encoding a pre-selected target protein (e.g., a reporter gene), is in proximity with a promoter, e.g., a tissue-specific promoter, to allow the promoter to regulate expression of the selected nucleic acid sequence. In general, the promoter is located upstream of the selected nucleic acid sequence in terms of the direction of transcription and translation. A HiNF-P binding site is operatively linked to a selected nucleic acid sequence when it is positioned relative to the selected sequence such that binding of HiNF-P increases expression of the selected sequence. In some cases, binding is inferred by assaying for an increase in expression of the selected sequence in the presence of HiNF-P expression compared to expression in the absence of HiNF-P expression.

A “promoter” is a nucleic acid sequence that directs transcription. A tissue-specific promoter induces expression of the selected nucleic acid sequence in specific cells, e.g., hematopoietic cells, or cells of a specific tissue within an animal, e.g., cardiac, muscle, or vascular endothelium. The term also covers so-called “leaky” promoters, which regulate expression of a selected nucleic acid sequence primarily in one tissue, but cause expression in other tissues as well. Such promoters also can include additional DNA sequences that are necessary for expression, such as introns and enhancer sequences.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference. In addition, the materials, methods, and examples are illustrative only and not intended to be limiting.

### DESCRIPTION OF DRAWINGS

FIG. 1A is a diagram of segments of NPAT (B42/N1, B42/N2, and B42/N3) used in preparing fusion expression constructs.

FIG. 1B is a representation of the results of yeast two hybrid experiments performed using the constructs illustrated in FIG. 1A and full-length HiNF-P.

FIG. 1C is a diagram of segments of HiNF-P used in preparing fusion expression constructs. The results of yeast two hybrid experiments with NPAT 1-533 are shown in the column to the right.

FIG. 1D is a diagram of segments of NPAT used in preparing fusion constructs. The results of yeast two hybrid experiments with full-length HiNF-P are shown in the column to the right. At the bottom of the figure is a depiction of the HiNF-P binding domains.

FIGS. 2A and 2B are diagrams and representations of immunoblots illustrating the results of co-immunoprecipitation experiments in HeLa (2A) and T98G (2B) cells.

FIGS. 2C and 2D are representations of immunoblots showing the results of co-immunoprecipitation experiments using 400 mg of Hela cell lysate which was subject to immunoprecipitation with the anti-NPAT antibody (2C) or using an anti-HiNF-P antibody with 400 mg of Hela cell lysate (2D).

FIG. 3 is a diagram and representation of an immunoblot showing the results of immunoprecipitation experiments using in vitro transcribed and translated (IVTT) NPAT and HiNF-P.

FIG. 4A is a representation of an autoradiogram showing the results of phosphoprotein analysis of HiNF-P (left lane: EV, HeLa cells transfected with an empty expression vector; right lane: FLAG-P, cells transfected with FLAG epitope-tagged HiNF-P expression vector).

FIG. 4B is a representation of an autoradiogram showing the results of phosphoprotein analysis of HiNF-P (λ Ppase, λ phosphatase treatment).

FIG. 4C is a representation of a Western blot using anti-FLAG-HRP, illustrating that the protein is still there, even after treatment with λ phosphatase.

FIG. 5A is a diagram of the Site II promoter sequence for cell cycle control of histone H4 gene transcription (SEQ ID NO:9). The H4 promoter is represented in the middle and illustrates two genomic sites of protein/DNA interactions (Site I and Site II) and cognate factors (SP1, ATI1, YY1). The three Site II proteins HiNF-P, -D, and -M recognize the overlapping recognition motifs (lower portion). Minimal element boundaries defined by DNase I footprinting and deletion analyses are indicated by the open or closed lines and protein/DNA contacts established by DMS fingerprinting are depicted by closed or open circles. The growth factor dependent CDK2/CLNE/NPAT pathway that functions to activate histone H4 gene transcription is also indicated (top).

FIG. 5B is a diagram of the histone H4 subtype specific consensus element (boldface) that is located upstream from the TATA box within Site II and was defined based on nine representative functionally expressed H4 genes (SEQ ID NOS:20-28).

FIG. 6A is an autoradiogram from a Site II competition assay testing the binding of HiNF-P to Site II double-stranded oligonucleotides (wild-type (H4/n, H4/e) and mutant (H4-Pmut)).

FIG. 6B is a representation of an autoradiogram showing UV cross-linking of HiNF-P/Site II complexes. Oligonucleotides spanning wild type (WT) and mutant (Pmut) Site II sequences were labeled with BrdUTP and ³²P-dCTP then incubated with a fraction (7.5 μl; 0.4M KCl) containing enriched HiNF-P binding activity. The reaction mixture was exposed to UV light (305 nm) for 60 minutes and then digested with DNase I and MNase. Cross-linked, labeled proteins were analyzed using 10% SDS-PAGE and subjected to autoradiography. Molecular weight makers are shown at the left. The arrow indicates the 65 kDa HiNF-P-DNA complex.

FIG. 6C is a representation of a silver-stained gel prepared by SDS-PAGE. The lanes contain aliquots of fractions collected from the Site II affinity column. Aliquots (125 μl) from each fraction of the DNA affinity step gradient were mixed with an equal volume of 2×SDS-PAGE sample buffer, loaded in 10% SDS-PAGE and stained with silver after electrophoresis. The two arrows at the bottom of the panel indicate the same 300 and 350 mM fractions exhibiting maximal HiNF-P binding activity in Panel B. Both fractions contain a unique 65 kDa band (indicated by horizontal arrowhead).

FIG. 6D is a diagram of the chromatographic separation procedure used to purify HiNF-P.

FIG. 7A is a schematic representation of a HiNF-P polypeptide. The domain structure of HiNF-P is based on the predicted translation of open reading frame in the HiNF-P cDNA. HiNF-P contains at least nine conserved C2H2 Zn-finger domains (gray shades). Peptide 1 (SEQ ID NO:11) is shown and was identified with MALDI-TOF mass spectrometric analysis of the purified HiNF-P polypeptide.

FIG. 7B is an autoradiogram of an SDS-PAGE experiment showing recombinant HiNF-P polypeptide synthesized by coupled in vitro transcription/translation (IVTT). ³⁵S-met labeled proteins were synthesized in a reticulocyte lysate system programmed with a vector containing HiNF-P coding sequences fused to an N-terminal epitope tag (Xpress™; Invitrogen). A 5 μl aliquot of the programmed lysate was mixed with an equal volume of 2× SDS-PAGE sample buffer and were subjected to 10% SDS-PAGE. The autoradiogram reveals the synthesis of a radiolabeled recombinant protein of the expected size (indicated by arrow).

FIG. 7C is an autoradiogram of an electrophoretic mobility shift assay (EMSA) with recombinant HiNF-P. Unlabeled recombinant HiNF-P with or without the Xpress tag was synthesized by IVTT and compared by EMSA analysis to endogenous HiNF-P in HeLa nuclear extracts (NE; first 3 lanes). Samples were subjected to oligonucleotide competition with wild type (W) or mutant (M) HiNF-P oligonucleotides or a non-specific oligonucleotide containing an SPI oligonucleotide (N). The Xpress™-tagged HiNF-P complex (X-P) migrates slower than the complexes formed with the untagged recombinant or endogenous HiNF-P.

FIG. 7D is an autoradiogram of an EMSA that shows that forced expression of HiNF-P increases HiNF-P binding activity. EMSA was performed with nuclear extract from. HeLa cells transiently transfected with HiNF-P cDNA (PCMV-HiNF-P). After 36 hours, cells were harvested, and nuclear protein (10 μg) from cells transfected with expression vector containing HiNF-P (+) or vector alone (−) was used for EMSA. Expression of HiNF-P elevates HiNF-P binding. C is a control binding reaction, W is a reaction in which excess wild type HiNF-P binding site oligonucleotide was used as a competitor, and M is a reaction in which excess mutant HiNF-P binding site oligonucleotide that cannot bind to HiNF-P was used as a competitor.

FIG. 7E is a Western blot showing that HiNF-P antipeptide antibody recognizes a 65 kD protein. HeLa cells were transfected with pCMV-HiNF-P (5 μg) or vector alone. After 36 hours, nuclear extracts were prepared and 20 μg of protein was prepared by 10% SDS-PAGE. Western blot analysis was performed with the affinity-purified antibody directed against Peptide 2 (CEKLQGIAEEPEIQMV (SEQ ID NO. 12); α802k; 1:1000). Both recombinant (+) and endogenous (−) HiNF-P are detected as 65 kD proteins (arrow).

FIG. 7F is a representation of a gel showing the results of an experiment monitoring the immunoreactivity of HiNF-P/Site II complexes. Antibodies directed against HiNF-P Peptide 1 (Anti-P #1) and Peptide 2 (Anti-P #2) and the corresponding pre-immune sera were incubated with endogenous HiNF-P from HeLa nuclear extracts complexed with a Site II oligonucleotide (P). HiNF-P binding activity is blocked by both antisera unless the matching peptides (Pep 1 and Pep 2, respectively) are present.

FIG. 8A is a representation of a gel (top) and a diagram illustrating the results of a ligation mediated (LM)-PCR analysis of HiNF-P binding sites in the histone H4 gene (bottom).

FIG. 8B is a representation of a gel showing the results of an experiment to detect the interaction between HiNF-P and the H4 promoter in vivo by chromatin immunoprecipitation using a HiNF-P antibody. Genomic segments derived from the H4 locus in chromatin precipitates from HL-60 cells were detected using PCR primers spanning 5′ (−219/+32) or 3′ (+644/+848) regions. Amplified products from the 5′ region are observed in input chromatin and in the precipitate with the HiNF-P antibody, but not in precipitates obtained with pre-immune-serum. The 3′ primer pair amplifies DNA only from input samples and serves as a negative control.

FIG. 9A is depiction of a Northern blot of an experiment showing histone H4 expression using total RNA isolated from proliferating (Prolif) and differentiated (Diff) HL-60 cells.

FIG. 9B is a photograph of the results of an EMSA demonstrating HiNF-P DNA binding activity (top) and protein levels (Western blot, bottom) in nuclear extracts from proliferating (d0) HL-60 cells and cells at various times after induction of differentiation with PMA (d1=1 day; d3=3 days).

FIG. 10A is a bar graph illustrating the results of experiments in which histone H4 transcription was assayed in the presence and absence of exogenous HiNF-P, NPAT, both, or neither in the presence and absence of an intact Site II sequence. The H4 promoter regions are diagramed at the top of the figure. Relative promoter activity (measured as luciferase activity) is shown as a function of the presence (+) or absence (−) of HiNF-P or NPAT expression vectors.

FIG. 10B is a bar graph illustrating the results of experiments in which histone H4 transcription was assayed in the presence and absence of exogenous HiNF-P, NPAT, both, or neither in the presence of a reporter gene that was operatively linked to a histone H4 promoter with multimerized HiNF-P (Site II) elements fused to a minimal TATA-box. The H4 promoter regions are diagramed at the top of the figure. Relative promoter activity (measured as luciferase activity) is shown as a function of the presence (+) or absence (−) of HiNF-P or NPAT expression vectors.

FIG. 11 is a bar graph illustrating the flow cytometry analysis of experiments in which cells were treated with anti-sense HiNF-P oligonucleotide, scrambled HiNF-P oligonucleotide, reverse oligonucleotide, or mock treated (control) for 30 hours then analyzed by flow cytometry for the percentage of cells in S phase. Each bar represents the data from 5 experiments. The asterisk indicates a statistical difference from control.

FIG. 12 is a diagram of the genomic locus of the mouse HiNF-P gene, including intron and exon structure and the nine conserved C2H2 zinc finger domains (numbers), promoter sequence, and start and stop codon placement.

FIGS. 13A and B are diagrams of the HiNF-P knock out construct targeting vector. FIG. 13A is a diagram showing the regions of the genomic sequence that were used for the left and right arms of the construct (which is illustrated in FIG. 13B).

FIG. 14A is a diagram illustrating the endogenous and targeted HiNF-P loci, showing the expected sizes after EcoR I digestion (4.5 and 6.3 kb, respectively), and the placement of probes A and C.

FIG. 14B is a representation of an autoradiogram showing the results of digestion of a number of ES cell clones with Eco RI. Each lane contains DNA from a different clone. Positive ES clones identified by Southern blot analysis are indicated with an asterisk.

FIG. 14C is a representation of a Southern confirming that each of the three distinct ES clones each contain only one targeted HiNF-P allele.

FIG. 15A is a diagram of HiNF-P domain structure, illustrating the Zinc-finger domain, glutamic acid-rich regions, and the C-terminal domain.

FIG. 15B is a diagram of two HiNF-P deletion mutants, Del 312-517 and Del 374-517.

FIG. 15C is a chart illustrating the activities of the HiNF-P wild type and deletion mutants.

FIG. 16 is a diagram illustrating the construction of a conditional HiNF-P knock out targeting vector, including the floxed Neomycin resistance gene (Neo). RA, right arm, MA, middle arm, LA, left arm. The small arrows illustrate the placement of sequencing primers.

FIG. 17 is a representation of the sequence of the LoxP cassette used in the construction of the conditional HiNF-P targeting vector, including a diagnostic XhoI site for genotyping and a SacII, EcoRV overhang (SEQ ID NOs:30 (forward) and 31 (reverse).

### DETAILED DESCRIPTION

As described herein, the nucleic acid and amino acid sequences associated with HiNF-P have been identified and HiNF-P activity has been characterized. HiNF-P was discovered to be a 65 kd zinc-finger protein that localizes to multiple, discrete subnuclear foci (Examples 2 and 5) and binds to Site II of the histone H4 gene locus (Examples 1 and 6). Levels of HiNF-P mRNA and protein were discovered to increase in proliferating cells and decrease when the cells become quiescent (Example 7). The increase of HiNF-P polypeptide in proliferating cells was found to correlate with increased HiNF-P binding to Site II promoter sequence along with an increase in H4 histone gene transcription (Example 8). Increased H4 histone gene transcription results in the packaging of newly synthesized DNA into chromatin and cell cycle progression from G1 to S. Thus, HiNF-P can be used to increase cell proliferation by inducing progression from G1 to S. HiNF-P can also be used as a target when screening for compounds that modulate cell proliferation, e.g., by decreasing the expression or activity of HiNF-P (for example, Example 10).

It has also been discovered that HiNF-P interacts directly with NPAT (nuclear protein mapped to the ATM locus; Example 9). ATM is a locus that is associated with ataxia-telangiectasia (A-T), an autosomal recessive disorder involving cerebellar degeneration, immunodeficiency, chromosomal instability, radiosensitivity, and a predisposition to cancer. NPAT is a substrate of the G1/S phase-related cyclin E/CDK2 kinase and acts as a co-activator of HiNF-P to enhance histone H4 gene transcription. The CDK2/cyclin E-HiNF-P-mediated phosphorylation sites of NPAT were discovered to be required for co-activating HiNF-P mediated histone H4 gene transcription; HiNF-P is the final link in the CDK2/cyclin E/NPAT pathway, coupling growth factor stimulation and the concomitant activation of this pathway with the induction of histone H4 expression at the G1/S phase transition. It was also discovered that multimers of Site II that are operatively linked to H4 can enhance induction of H4 transcription by HiNF-P.

HiNF-P

As described herein, a sequence has been identified in a human nucleic acid database that corresponds to full-length HiNF-P cDNA. The sequence was previously identified as an expressed sequence in human lung small cell carcinoma (Genbank accession No. BC017234; SEQ ID NO:5). This HiNF-P cDNA clone was identified in the database screen based on data obtained using the purification strategy described in the Examples and sequencing the putative HiNF-P polypeptide that was isolated. The coding sequence for HiNF-P contains the identified 2.1 kb cDNA. Oligonucleotide primers can be used to amplify an approximately 1.5 kb DNA segment spanning the entire open reading frame by RT-PCR using total HeLa mRNA (see, e.g., Example 3).

NPAT

NPAT was previously identified as an important alternate substrate of Cdk2/cyclin E, which plays an important role in promoting cell-cycle progression and contributes to cell-cycle regulated histone gene expression. The nucleic acid sequence of NPAT is Genbank accession no. U58852; SEQ ID NO:7. The amino acid sequence of NPAT is Genbank Accession no. AAB02735; SEQ ID NO:8. As demonstrated herein, NPAT interacts with HiNF-P directly, at the N-terminus (see Example 8). The invention includes fragments of NPAT that bind to HiNF-P, e.g., fragments including the first 533 amino acids of NPAT, e.g., amino acids 1-46 and/or amino acids 220-330, or a portion thereof, and fragments of HiNF-P that bind to NPAT, e.g., fragments including the first 230 amino acids of HiNF-P, or a portion thereof.

Site II Genomic DNA

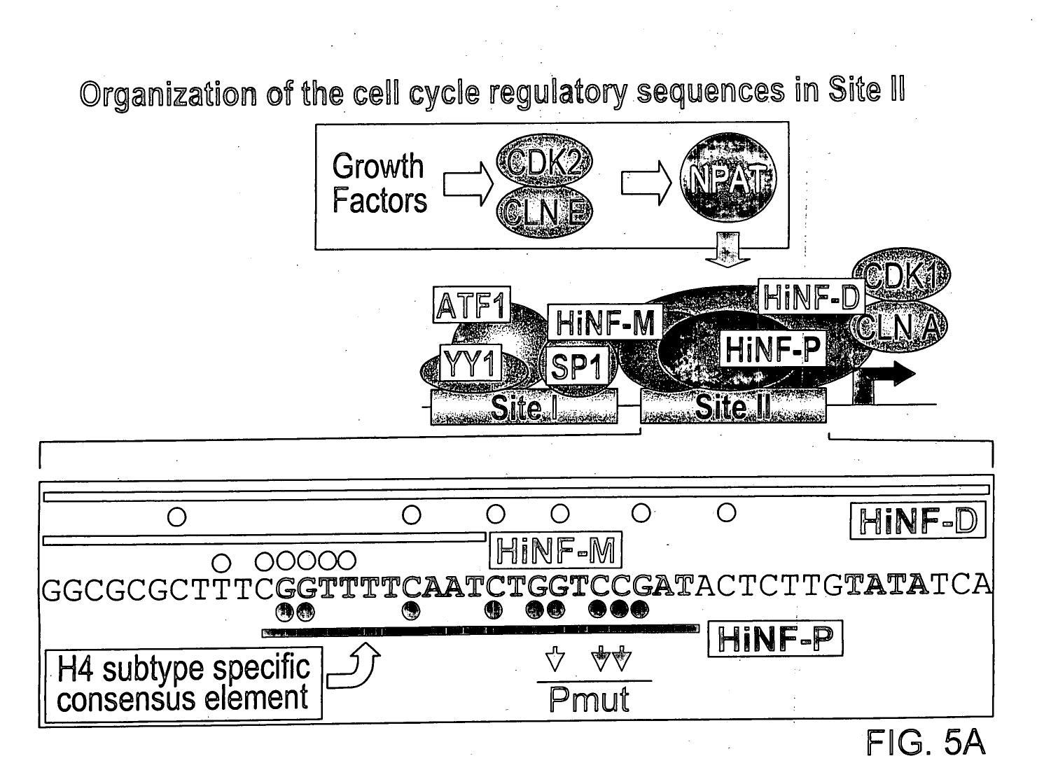

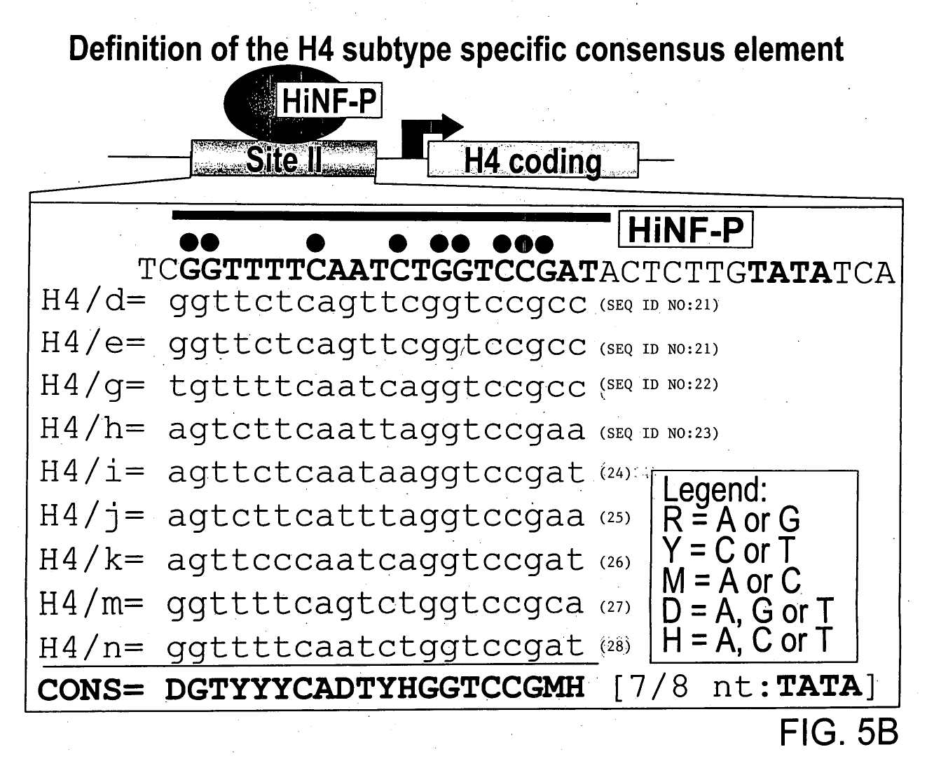

The H4-Site II transcriptional element of H4 histone genes is a proximal promoter element that interacts with at least three distinct sequence-specific DNA binding activities, HiNF-D, HiNF-M, and HiNF-P. HiNF-D binds to an extended series of nucleotides, whereas HiNF-M and HiNF-P recognize sequences internal to the HiNF-D binding domain. FIG. 5A illustrates the Site II promoter sequence for cell cycle control of histone H4 gene transcription. Panel A diagrams the H4 promoter (middle) and shows two genomic sites of protein/DNA interactions (Site I and Site II) and the cognate factors (e.g., SP1, ATI1, YY1). The three Site II proteins HiNF-P, -D and -M recognize overlapping recognition motifs (lower portion). Minimal element boundaries defined by DNase I footprinting and deletion analyses are indicated by the open or closed lines in FIG. 5A and protein/DNA contacts that were established by DMS fingerprinting are depicted by closed or open circles. The growth factor dependent CDK2/CLNE/NPAT pathway that functions to activate histone H4 gene transcription is also indicated (top).

The key factor that interacts with the Site II cell cycle regulatory element (FIG. 5A) is HiNF-P, which binds to the H4 subtype specific sequence that is highly conserved among the fourteen H4 gene copies present in the human genome (FIG. 5B). A Site II consensus sequence is shown in FIG. 5B (SEQ ID NO: 20); in bold is a minimal consensus sequence (5′-GGTTTTCAATCTGGTCCGAT-3′; SEQ ID NO:10).

The invention includes methods of isolating HiNF-P peptides. Such methods can include using a Site II sequence on an affinity column as illustrated in the Examples below. The sequence is also useful in screening assays to identify compounds that modulate HiNF-P binding to Site II. Examples of such assays are provided below.

The invention also encompasses methods of enhancing transcription of H4 by inserting multiple copies of Site II (e.g., consensus sequence) so that they are operatively linked to a transcribed histone H4 sequence, a non-histone H4 sequence (e.g., a reporter gene), or a hybrid sequence comprising at least part of a histone H4 sequence and sequence that is not histone H4. Furthermore, by operatively linking Site II sequence (one or more copies, for example 2-10 copies) to a heterologous sequence, increased transcription of the heterologous sequence can be induced by HiNF-P that is either endogenous to a cell in which the engineered heterologous sequence is located or by HiNF-P that is introduced by genetically engineering the cell (e.g., with an expression vector as described below). Methods for engineering a cell to include multimerized Site II are known in the art, as are methods of introducing exogenous sequence (e.g., HiNF-P).

HiNF-P Polypeptides

Three-frame translation analysis revealed an open reading frame of the cDNA that encodes a zinc finger protein with a predicted size of 517 amino acids. The amino acid sequence of HiNF-P is Genbank accession no. AAH17234; SEQ ID NO:6.

Isolated HiNF-P polypeptides, or a fragment thereof, are useful in some embodiments. For example, a HiNF-P polypeptide or fragment thereof can be used as an antigen to produce antibodies that specifically bind to HiNF-P. HiNF-P peptides are also useful for testing the binding properties of antibodies raised against a HiNF-P polypeptide. Furthermore, isolated HiNF-P polypeptides are useful in in vitro screening assays, e.g., to identify compounds that specifically bind to HiNF-P, inhibit HiNF-P interactions, or inhibit HiNF-P-NPAT interactions.

A HiNF-P polypeptide can be isolated from cells or tissue sources using protein purification techniques that are known in the art, some of which are exemplified below. HiNF-P polypeptides or fragments thereof can also be produced by recombinant DNA techniques or synthesized chemically.

In general, a HiNF-P polypeptide is characterized by one or more of the following:

(i) it has the ability to enhance H4 histone gene transcription;

(ii) it has a molecular weight, e.g., a deduced molecular weight (generally excluding post-translational modifications), amino acid composition, or other physical characteristic substantially similar to a polypeptide of SEQ ID NO:6;

(iii) it has an overall sequence similarity of at least about 50% with a polypeptide of SEQ ID NO:6. In some embodiments, it has an overall similarity of at least about 60%, 70%, 80%, 90%, or 95%;

(iv) it is localized to the nucleus when expressed in a cell;

(v) it binds to NPAT;

(vi) it specifically binds to Site II nucleic acid sequence; and/or

(vii) it enhances H4 histone gene transcription when co-expressed with NPAT.

The HiNF-P polypeptide, or fragment thereof, can differ from the corresponding sequence in SEQ ID NO:6. For example, such a polypeptide can differ by more than one but less than about 15 amino acid residues. In some embodiments, the polypeptide differs from SEQ ID NO: 6 by less than about 10 or 5 amino acid residues. In other cases, the polypeptide differs from the corresponding sequence or fragment thereof in SEQ ID NO:6 by at least one residue, but less than about 20% (e.g., in some embodiments, less than about 15%, 10% or 5%) of the residues in it differ from the corresponding sequence in SEQ ID NO:6. (If this comparison requires alignment the sequences should be aligned for maximum homology. “Looped out” sequences from deletions or insertions, or mismatches, are considered differences.) The differences are, typically, at a non-essential residue or are conservative substitutions.

A useful HiNF-P polypeptide can contain changes in a portion of the amino acid sequence that is not essential for activity, e.g., a change that is not in the regions of HiNF-P that are required for binding to Site II. Such HiNF-P polypeptides differ in amino acid sequence from SEQ ID NO:6, and yet retain biological activity.

The protein can include an amino acid sequence at least about 70%, 75%, 80%, 85%, 90%, 95%, 98% or more homologous or identical to SEQ ID NO:6.

HiNF-P Fusion Proteins

A HiNF-P polypeptide can be expressed as a chimeric protein or fusion protein. As used herein, a HiNF-P “chimeric protein” or “fusion protein” includes a HiNF-P polypeptide linked to a non-HiNF-P polypeptide. A “non-HiNF-P polypeptide” refers to a polypeptide having an amino acid sequence corresponding to a protein that is not substantially homologous to the HiNF-P polypeptide, e.g., a protein which is different from the HiNF-P polypeptide and which is derived from the same or a different organism. Such fusion proteins are useful, e.g., for detecting and/or purifying HiNF-P in various methods, such as immunocytochemical methods in which the non-HiNF-P portion of the chimeric protein is detected.

The HiNF-P polypeptide of the fusion protein can correspond to all or a portion, e.g., a fragment described herein of a HiNF-P amino acid sequence. In general, a HiNF-P fusion protein includes at least one (e.g., two) biologically active portions of a HiNF-P polypeptide, e.g., the Site II binding portion of HiNF-P. The non-HiNF-P polypeptide portion of the fusion protein can be fused to the N-terminus or C-terminus of the HiNF-P polypeptide.

In some embodiments, the fusion protein can include a moiety that has a high affinity for a ligand. For example, the fusion protein can be a GST-HiNF-P fusion protein in which the HiNF-P sequences are fused to the C-terminus of the GST sequences. Such fusion proteins can facilitate the purification of recombinant HiNF-P. Alternatively, the fusion protein can be a HiNF-P polypeptide containing a heterologous signal sequence at its N-terminus. In certain host cells (e.g., mammalian host cells), expression and/or secretion of HiNF-P can be increased through use of a heterologous signal sequence.

A nucleic acid sequence encoding a HiNF-P fusion protein can be incorporated into a pharmaceutical composition and administered to a subject in vivo. Such a HiNF-P fusion protein can be used to affect the bioavailability of a HiNF-P substrate, for example, by binding to Site II, thereby inhibiting binding of endogenous HiNF-P to Site II. If the HiNF-P fusion protein can bind to Site II, but does not promote histone H4 transcription, the fusion protein can be useful for treating disorders associated with increased cellular proliferation such as a cancer. The invention includes methods of screening for compounds such as fusion protein that modulates histone H4 expression by binding to a HiNF-P binding site.

HiNF-P-fusion proteins can also be used as immunogens to produce anti-HiNF-P antibodies, to purify HiNF-P ligands, and in screening assays to identify molecules that enhance or inhibit the interaction of HiNF-P with a HiNF-P substrate.

Expression vectors are commercially available that already include a sequence encoding a fusion moiety (e.g., a GST polypeptide). A HiNF-P-encoding nucleic acid can be cloned into such an expression vector such that the fusion moiety is linked in-frame to the HiNF-P polypeptide using known methods.

Recombinant Expression Vectors, Host Cells, and Genetically Engineered Cells

Vectors, e.g., expression vectors, containing a nucleic acid encoding a polypeptide are useful in some embodiments of the invention. As used herein, the term “vector” refers to a nucleic acid molecule that can another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a HiNF-P nucleic acid in a form suitable for expression of the nucleic acid in a host cell. In general, the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., HiNF-P polypeptides, mutant forms of HiNF-P polypeptides, fusion proteins, and the like).

The recombinant expression vectors of the invention can be designed for expression of HiNF-P polypeptides in prokaryotic or eukaryotic cells. For example, a HiNF-P polypeptide can be expressed in E. coli, insect cells (e.g., using baculovirus expression vectors), yeast cells, or mammalian cells. Suitable host cells are known in the art, e.g. Goeddel, (Gene Expression Technology: Methods in Enzymology, 185 (Academic Press, San Diego, Calif. (1990)). Alternatively, the recombinant expression vector can be transcribed and translated in vitro, for example using T7 promoter regulatory sequences and T7 polymerase.

Expression of proteins in prokaryotes is generally carried out in E. coli with vectors containing constitutive or inducible promoters directing the expression of either fusion or non-fusion HiNF-P polypeptides. Fusion vectors are used to produce fusion proteins such as those described supra. They add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, and enterokinase. Typical fusion expression vectors include pGEX (Pharmacia Biotech Inc; 1998, Smith et al. Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used, e.g., in HiNF-P activity assays, (e.g., direct assays or competitive assays described herein), or to generate antibodies specific for HiNF-P polypeptides. In some cases, a fusion protein expressed in a retroviral expression vector can be used to infect bone marrow cells that are subsequently transplanted into irradiated recipients. The pathology of the subject recipient is then examined after sufficient-time has passed to permit an expected effect of the fusion protein, e.g., a therapeutic effect (e.g., six weeks).

To maximize recombinant protein expression in E. coli, a HiNF-P polypeptide can be expressed in a host bacterium with an impaired capacity to proteolytically cleave the recombinant protein (Gottesman, Gene Expression Technology: Methods in Enzymology 185 (Academic Press, San Diego, Calif., 1990) pp. 119-128). Another strategy is to alter the sequence of the nucleic acid to be inserted into an expression vector so that the individual codons for each amino acid are those preferentially utilized in E. coli (Wada et al., Nucleic Acids Res., 20:2111-2118, 1992). Such alteration of nucleic acid sequences of the invention can be carried out using DNA mutagenesis and/or synthesis techniques that are known in the art.

A HiNF-P expression vector can be a yeast expression vector, a vector for expression in insect cells, e.g., a baculovirus expression vector, or a vector suitable for expression in mammalian cells. When used in mammalian cells, the expression vector's control functions can be provided by viral regulatory elements. Examples include promoters derived from polyoma, adenovirus 2, cytomegalovirus (CMV), and Simian Virus 40 (SV40).

A recombinant mammalian expression vector that is useful can direct expression of the nucleic acid preferentially in a particular cell type (e.g., tissue-specific regulatory elements are used to express the nucleic acid). Non-limiting examples of suitable tissue-specific promoters include the albumin promoter that is liver-specific (Pinkert et al., 1987, Genes Dev., 1:268-277), lymphoid-specific promoters (Calame and Eaton, 1988, Adv. Immunol., 43:235-275) such as promoters of T cell receptors (Winoto and Baltimore, 1989, EMBO J., 8:729-733) and immunoglobulins (Banerji et al., 1983, Cell, 33:729-740; Queen and Baltimore, 1983, Cell, 33:741-748). Useful tissue-specific promoters can also be neuron-specific promoters (e.g., the neurofilament promoter; Byrne and Ruddle, 1989, Proc. Natl. Acad. Sci. USA, 86:5473-5477), pancreas-specific promoters (Edlund et al., 1985, Science, 230:912-916), and mammary gland-specific promoters (e.g., milk whey promoter; U.S. Pat. No. 4,873,316 and European Application Publication No. 264,166). Developmentally regulated promoters can also be used, for example, a murine hox promoter (Kessel and Gruss, 1990, Science, 249:374-379) and the α-fetoprotein promoter (Campes and Tilghman, 1989, Genes Dev., 3:537-546). The vector can also contain sequences known in the art that will cause the secretion of the HinF-P polypeptide, e.g., in the milk of the mammal.

The recombinant expression vector can also encode an antisense molecule that is useful for inhibiting expression of a HiNF-P polypeptide or NPAT. Such methods are known in the art, e.g., Weintraub et al., 1986, Reviews—Trends in Genetics, 1:1. Regulatory sequences (e.g., viral promoters and/or enhancers) operatively linked to a nucleic acid cloned in the antisense orientation can be chosen, which direct the constitutive, tissue specific, or cell-type specific expression of antisense RNA in a variety of cell types. The antisense expression vector can be in the form of a recombinant plasmid, phagemid, or attenuated virus.

Some embodiments relate to a host cell that includes a nucleic acid molecule described herein, e.g., a HiNF-P nucleic acid molecule within a recombinant expression vector, or a HiNF-P nucleic acid molecule containing sequences that allow it to homologously recombine into a specific site of the host cell's genome. The terms “host cell” and “recombinant host cell” are used interchangeably herein. Such terms refer not only to a cell in a particular subject, but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a HiNF-P polypeptide can be expressed in a bacterial cell such as E. coli, an insect cell, yeast, or mammalian cell (such as Chinese hamster ovary cell or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell can be used to produce (i.e., express) a HiNF-P polypeptide. In one embodiment, the host cell (into which a recombinant expression vector encoding a HiNF-P polypeptide has been introduced) is cultured in a suitable medium such that a HiNF-P polypeptide is produced. In another embodiment, the method further includes isolating a HiNF-P polypeptide from the medium or the host cell.

A cell or purified preparation of cells can include cells comprising a HiNF-P transgene, or cells that otherwise misexpress (e.g., over- or under-express) HiNF-P. The cell preparation can consist of human or non-human cells, e.g., rodent cells such as mouse or rat cells, rabbit cells, or pig cells. In some cases, the cell or cells include a HiNF-P transgene, e.g., a heterologous form of a HiNF-P, e.g., a gene derived from humans (in the case of a non-human cell). The HiNF-P transgene can be normally expressed or misexpressed, e.g., over-expressed or under-expressed. In other embodiments, the cell or cells include a gene that misexpresses an endogenous HiNF-P, e.g., a gene the expression of which is disrupted, e.g., a knockout. Such cells can serve as a model for studying disorders that are related to mutated or mis-expressed HiNF-P alleles or for use in screening assays for compounds that modulate HiNF-P expression or activity.

A cell containing an endogenous HiNF-P, under the control of an exogenous regulatory sequence is also useful. Such cells are generally cells in which it is desirable to control expression of HiNF-P. For example, in a useful cell of this type, the expression characteristics of an endogenous gene within a cell, e.g., a cell line or microorganism, can be modified by inserting a heterologous DNA regulatory element into the genome of the cell such that the inserted regulatory element is operably linked to the endogenous HiNF-P gene. For example, an endogenous HiNF-P gene that is “transcriptionally silent,” e.g., not normally expressed, or expressed only at very low levels, can be activated by inserting a regulatory element which is capable of promoting the expression of a normally expressed gene product in that cell. Known techniques, such as targeted homologous recombination, can be used to insert the heterologous DNA as described in, e.g., Chappel, U.S. Pat. No. 5,272,071; or WO 91/06667, published May 16, 1991.

Antisense Nucleic Acid Molecules, Ribozymes, and Modified HiNF-P Nucleic Acid Molecules

In some embodiments, the invention includes an isolated nucleic acid molecule that is antisense to HiNF-P. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire HiNF-P coding strand, or to only a portion thereof (e.g., the coding region of human HiNF-P corresponding to nucleotide positions 41-60, 51-76, 1240-1259, or 1454-1473 in the HiNF-P cDNA of SEQ ID NO:5 (Genbank Accession No. BC017234). In another embodiment, the antisense nucleic acid molecule is antisense to a “noncoding region” of the coding strand of a nucleotide sequence encoding HiNF-P (e.g., the 5′ and 3′ untranslated regions).

In general, an antisense oligonucleotide is antisense to only a portion of the coding or noncoding region of a HiNF-P mRNA. For example, the antisense oligonucleotide can be complementary to the region surrounding the translation start site of HiNF-P mRNA, e.g., between the −10 and +10 regions of the target nucleotide sequence of interest. An antisense oligonucleotide can be, for example, at least about 7 nucleotides in length. In some embodiments, the antisense oligonucleotide is, or is at least, about 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

An “anti-sense oligonucleotide specific for HiNF-P” or a “HiNF-P anti-sense oligonucleotide” is an oligonucleotide having a sequence (a) that can form a stable triplex with a portion of the HiNF-P gene, or (b) can form a stable duplex with a portion of an mRNA transcript of the HiNF-P gene. For example, 5′-GGGCATTGGTCTGATTCACC-3′ (SEQ ID NO:17) has been shown to be an effective antisense oligonucleotide for HiNF-P.

An antisense nucleic acid molecule of the invention can be administered to a subject (e.g., by direct injection at a tissue site), or generated in situ such that it hybridizes with or binds to cellular mRNA and/or genomic DNA encoding a. HiNF-P polypeptide, to thereby inhibit expression of the protein, e.g., by inhibiting transcription and/or translation. Alternatively, antisense nucleic acid molecules can be modified to target selected cells and then administered systemically. In some embodiments when systemic administration is desired, the antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are generally used.

An antisense nucleic acid molecule can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res., 15:6625-6641). The antisense nucleic acid molecule can also comprise a 2′-o-methylribonucleotide (Inoue et al., 1987, Nucleic Acids Res. 15:6131-6148) or a chimeric RNA-DNA analog (Inoue et al., 1987, FEBS Lett. 215:327-330).

A ribozyme having specificity for a HiNF-P-encoding nucleic acid can also be used to regulate HiNF-P expression. Such a ribozyme generally includes one or more sequences complementary to the nucleotide sequence of a HiNF-P cDNA (i.e., SEQ ID NO:5), and a sequence having a known catalytic sequence responsible for mRNA cleavage (see U.S. Pat. No. 5,093,246; Haselhoff and Gerlach, 1988, Nature, 334:585-591). For example, a derivative of a Tetrahymena L-19 IVS RNA can be constructed in which the nucleotide sequence of the active site is complementary to the nucleotide sequence to be cleaved in a HiNF-P-encoding mRNA (e.g., Cech et al., U.S. Pat. No. 4,987,071; and Cech et al., U.S. Pat. No. 5,116,742). Alternatively or in addition, HiNF-P mRNA can be used to select a catalytic RNA having a specific ribonuclease activity from a pool of RNA molecules (e.g., Bartel et al., 1993, Science 261:1411-1418.

Cells can be treated with an anti-sense oligonucleotide by mixing the cells with the oligonucleotide and subjecting the mix to standard transformation protocols (e.g., electroporation or liposome fusion).

HiNF-P gene expression can be inhibited by targeting nucleotide sequences complementary to the regulatory region of the HiNF-P (e.g., the HiNF-P promoter and/or enhancers) to form triple helical structures that prevent transcription of the HiNF-P gene in target cells (e.g., Helene, 1991, Anticancer Drug Des., 6:569-84; Helene, 1992, Ann. N.Y. Acad. Sci., 660:27-36; and Maher, 1992, Bioassays, 14:807-15). The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Detectably labeled oligonucleotide primer or probe molecules are also useful. Typically, such labels are chemiluminescent, fluorescent, radioactive, or colorimetric.

A HiNF-P nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al., 1996, Bioorganic & Medicinal Chemistry, 4:5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols, e.g., as described in Hyrup et al., 1996, supra and Perry-O'Keefe et al., Proc. Natl. Acad. Sci., 93: 14670-675.

PNAs of HiNF-P nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of HiNF-P nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al., 1996, supra)); or as probes or primers for DNA sequencing or hybridization (Hyrup B. et al., 1996, supra; Perry-O'Keefe supra).

The HiNF-P oligonucleotides can include other appended groups such as peptides (e.g., for targeting host cell receptors in vivo), or agents facilitating transport across the cell membrane (see, e.g., Letsinger et al. (1989) Proc. Natl. Acad. Sci. USA, 86:6553-6556; Lemaitre et al. (1987) Proc. Natl. Acad. Sci. USA, 84:648-652; PCT Publication No. WO88/09810) or the blood-brain barrier (see, e.g., PCT Publication No. WO89/10134). In addition, HiNF-P oligonucleotides can be modified with hybridization-triggered cleavage agents (see, e.g., Krol et al. (1988) Bio-Techniques, 6:958-976) or intercalating agents. (see, e.g., Zon (1988) Pharm. Res., 5:539-549). To this end, the HiNF-P oligonucleotides can be conjugated to another molecule, (e.g., a peptide, hybridization triggered cross-linking agent, transport agent, or hybridization-triggered cleavage agent).

Molecular beacon oligonucleotide primer and probe molecules having at least one region that is complementary to a HiNF-P nucleic acid are also useful. In general, a molecular beacon molecule having two complementary regions, one having a fluorophore and one a quencher, is useful for quantitating the presence of the HiNF-P nucleic acids described herein in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Modulation of HiNF-P Activity

In some embodiments, HiNF-P activity is modulated by increasing or decreasing the levels of active HiNF-P polypeptide in a cell-free system or within a cell, e.g., in vitro or in an animal such as a mammal. Also, HiNF-P activity can be modulated by altering one or more of HiNF-P's biochemical activities, e.g., by contacting HiNF-P with cofactors or small molecules that interact with HiNF-P.

Enhancing HiNF-P's activity promotes cell growth and cell cycle progression through S phase. Enhancement of HiNF-P activity can be used for the treatment of conditions that would benefit from increased cellular proliferation, such as unhealed wounds, or when increased tissue or organ growth or regrowth, or angiogenesis is desired, e.g., by specifically promoting the growth of certain cells, e.g., progenitors along different cell lineages. Enhancement of HiNF-P activity can be used to modulate the ex vivo expansion of cells used for culturing tissues (e.g., culturing cells for skin grafts) and organs, or for gene therapy. Specific promoters or conditional promoters can be used to control cellular proliferation when suitable.

The purpose of inhibiting HiNF-P activity is to inhibit cell proliferation at the G1/S phase transition, or to uncouple histone gene expression and DNA replication. Agents that block HiNF-P activity can be used for therapeutic intervention in proliferation-related diseases (e.g., cancer of soft and hard tissues, lymphomas, leukemias, and psoriasis), for regulating ex vivo expansion of cells and tissues (e.g., bone marrow, stem cells, and pre-committed progenitor cells), and for gene therapy.

Modulation of HiNF-P Activity by Over-Expression of Recombinant HiNF-P

HiNF-P activity is increased by increasing the amount of HiNF-P polypeptide in a cell by introducing into the cell a vector including a sequence encoding a HiNF-P polypeptide that can be translated in the cell. Useful expression vectors include a constitutive expression vector, an inducible expression vector, or a tissue specific expression vector. A sequence encoding a HiNF-P polypeptide can be operatively linked to the promoter of an expression vector and introduced into a cell. The promoter can express the HiNF-P sequence constitutively, inducibly, or in a tissue specific manner above the normal levels of endogenous HiNF-P found in a cell. HiNF-P production can be monitored, e.g., by Northern blot analysis, PCR, or immunoprecipitation.

A defective HiNF-P DNA sequence encodes a HiNF-P polypeptide that can bind a Site II promoter sequence without activating H4 histone gene transcription. For example, a HiNF-P polypeptide that binds cellular modulators of HiNF-P, but can no longer bind to Site II promoter sequence, can be used as a dominant-negative. A dominant-negative effectively dilutes out the activity of the endogenously expressed HiNF-P polypeptide. The mutant HiNF-P nucleic acid sequence is operatively linked to a promoter sequence in an expression vector as discussed above, and introduced into a cell. The over-expressed, defective HiNF-P polypeptide can then override the activity of the normal, endogenous HiNF-P. In some embodiments, the dominant negative form of HiNF-P cannot be phosphorylated, e.g., contains a mutation at one or more consensus phosphorylation sites, e.g., at one or both of the threonine or serine resides present in the highly conserved YESVELTE sequence (SEQ ID NO:32).

Exogenous normal or defective HiNF-P over-expression can be monitored by measuring the enhancement or inhibition, respectively, of H4 histone gene transcription. H4 histone gene transcription can be monitored by Northern blot analysis or PCR. Also, increased or decreased levels of H4 histone protein resulting from increased levels of active or defective HiNF-P polypeptide expression, and can be measured, e.g., by immunoprecipitation of the H4 histone protein.

Increased or decreased levels of active or defective HiNF-P polypeptide in a cell can be measured with assays that monitor G1/S phase transition by monitoring DNA synthesis using the incorporation of [³H]thymidine into DNA as a marker. For example, cells that are to be assayed are incubated in the presence of [³H]thymidine, harvested onto filters, and washed with trichloroacetic acid. The DNA retained on the filters is quantified by counting the filters in a liquid scintillation cocktail. Increased DNA synthesis compared to a control indicates the presence of an active, exogenous HiNF-P polypeptide, where decreased DNA synthesis compared to a control indicates the presence of an inactive HiNF-P polypeptide. In general, the control is a normal cell from which the cell being assayed is derived.

The enhancing or inhibiting activity of normal or defective HiNF-P can also be measured by monitoring the G1/S phase transition by labeling the cells with the thymidine analogue 5-bromo-2′-deoxy-uridine (BrdU). BrdU is incorporated into cellular DNA in place of thymidine. The incorporated BrdU can be detected by a quantitative cellular enzyme immunoassay using monoclonal antibodies directed against BrdU.

NPAT can be co-expressed with active HiNF-P in the same cell. The presence of over-expressed NPAT and HiNF-P in the same cell results in greater enhancement of H4 histone transcription relative to the presence of either of the over-expressed proteins alone. The sequences encoding both proteins can be placed in the same expression vector or in separate vectors. If the cDNA sequences are placed in the same vector, they can be placed head-to-tail and transcription can initiate from a single promoter and move in a single direction. They can also be placed head to head with a separate promoter driving transcription in opposite directions.

The sequences encoding HiNF-P polypeptide and NPAT polypeptide can be inserted into individual vectors and sequentially introduced into the cell. In general, each vector will also contain a different drug resistance gene, thus, cells containing both vectors can be selected by exposing the cells to both drugs to which the vectors confer resistance.

When HiNF-P and NPAT are used to modulate enhanced H4 histone transcription in concert, the promoters used to transcribe the sequences encoding the polypeptides can be the same or different (e.g., one can choose an inducible promoter, a constitutive promoter, or a tissue specific promoter for each vector (both different or the same).

The ability of the sequences encoding HiNF-P and NPAT polypeptides to be expressed can be assayed using methods described herein and those known in the art.

Screening Assays for Modulators of HiNF-P Expression or Activity

In some embodiments, the invention includes compounds that enhance or inhibit HiNF-P expression or activity. Sources of test compounds that may have such properties (candidate compounds) include combinatorial libraries, defined chemical identities, libraries of peptides and/or peptide mimetic, oligonucleotides, or antibodies, and natural product libraries. These can be screened for activity as enhancers and inhibitors of HiNF-P expression or activity in assays such as those described herein. Test compounds that show activity in assays such as those described below are referred to as candidate compounds (e.g., inhibitors), which can be tested in in vivo systems, such as an animal model. Candidate inhibitors can be tested for their ability to attenuate uncontrolled cell proliferation and candidate enhancers can be tested for their ability to enhance cell proliferation in an animal model.

The invention provides screening methods (also referred to herein as “assays”) for identifying test compounds (e.g., proteins, peptides, peptidomimetics, peptoids, nucleic acids, aptamers, carbohydrates, polysaccharides, non-nucleic acid small organic molecules, inorganic molecules, and drugs) that bind to a HiNF-P polypeptide, have an inhibitory or stimulatory effect on, for example, HiNF-P expression (protein or nucleic acid) or HiNF-P activity, or have a stimulatory or inhibitory effect on, for example, the expression or activity of a HiNF-P substrate (e.g., a Site II promoter sequence, or a gene driven thereby). Candidate compounds thus identified can be used to modulate the activity of a HiNF-P gene or gene product either directly or indirectly, to elaborate the biological function of a HiNF-P gene product, or to identify compounds that disrupt normal HiNF-P gene-gene, protein-gene, or protein-protein interactions. Candidate compounds that inhibit the activity or expression of HiNF-P can be useful as inhibitors of cell proliferation, e.g., useful in the treatment of disorders characterized by cellular proliferation, e.g., cancers such as carcinomas.

In some embodiments, the invention provides assays for screening test compounds for their ability to bind to or modulate the activity of a HiNF-P polypeptide or a biologically active portion thereof.

Compounds to be screened (“test compounds”) can be obtained using any of the numerous approaches in combinatorial library methods known in the art. Such libraries include biological libraries; peptoid libraries (libraries of molecules having the functionalities of peptides, but with a novel, non-peptide backbone, which are resistant to enzymatic degradation, but which nevertheless remain bioactive, (e.g., as described in Zuckermann et al., 1994, J. Med. Chem. 37:2678-2685)), or spatially addressable parallel solid phase or solution phase libraries. Methods to make such libraries include synthetic library methods requiring deconvolution, the ‘one-bead one-compound’ library method, and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993, Proc. Natl. Acad. Sci. U.S.A., 90:6909; Erb et al. (1994, Proc. Natl. Acad. Sci. USA, 91:11422); Zuckermann et al. (1994, J. Med. Chem., 37:2678, Cho et al. (1993, Science, 261:1303; Carrell et al. (1994, Angew. Chem. Int. Ed. Engl., 33:2059); Carell et al. (1994, Angew. Chem. Int. Ed. Engl., 33:2061); and Gallop et al. (1994, J. Med. Chem., 37:1233).

Libraries of compounds can be presented in solution (e.g., Houghten, 1992, Biotechniques, 13:412-421), or on beads (Lam, 1991, Nature, 354:82-84), chips (Fodor, 1993, Nature, 364:555-556), bacteria (Ladner, U.S. Pat. No. 5,223,409), spores (Ladner, U.S. Pat. No. 5,223,409), plasmids (Cull et al., 1992, Proc. Natl. Acad. Sci. USA, 89:1865-1869) or on phage (Scott and Smith, 1990, Science, 249:386-390; Devlin, 1990, Science, 249:404-406; Cwirla et al., 1990, Proc. Natl. Acad. Sci, 87:6378-6382; Felici, 1991, J. Mol. Biol., 222:301-310; and Ladner supra.).

In certain embodiments, the screening methods are cell-based assays in which a cell that expresses a HiNF-P polypeptide or biologically active portion thereof is contacted with a test compound, and the ability of the test compound to modulate HiNF-P activity is determined. Determining the ability of the test compound to modulate HiNF-P activity can be accomplished by monitoring, for example, changes in the ability of HiNF-P to interact with a Site II promoter sequence or NPAT. Such assays can be performed using a cell (e.g., a cell that naturally contains the Site II promoter sequence or NPAT, or a cell into which the Site II promoter sequence has been inserted, (e.g., with or without a reporter gene) or the Site II promoter sequence in solution (in vitro). The ability of the test compound to modulate HiNF-P binding to the Site II promoter sequence, or to bind to HiNF-P can be evaluated. This can be accomplished, for example, by coupling the test compound (or NPAT (or a fragment thereof) or a polynucleotide comprising the Site II promoter sequence) with a radioisotope or enzymatic label such that binding of the compound (or NPAT or the Site II promoter sequence) to HiNF-P can be determined by detecting the labeled compound, e.g., substrate, in a complex. Alternatively, HiNF-P can be coupled with a radioisotope or enzymatic label to monitor the ability of a test compound to modulate HiNF-P binding to a HiNF-P substrate such as Site II promoter sequence. For example, the test compounds, the HiNF-P, or the Site II promoter sequence can be labeled with ¹²⁵I, ³⁵S, ¹⁴C, or ³H, either directly or indirectly, and the radioisotope detected by standard methods, e.g., direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a test compound to interact with HiNF-P with or without the labeling of any of the interactants can be evaluated. For example, a microphysiometer can be used to detect the interaction of a compound with HiNF-P without labeling either the compound or the HiNF-P (McConnell et al., 1992, Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and HiNF-P.

In some embodiments, a cell-free assay is used in which a HiNF-P polypeptide or biologically active portion thereof is contacted with a test compound, and the ability of the test compound to bind to the HiNF-P polypeptide or biologically active portion thereof is evaluated. Biologically active portions of the HiNF-P polypeptides that can be used in such assays include fragments that participate in interactions with non-HiNF-P molecules (such as NPAT or a Site II promoter sequence), e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules (e.g., HiNF-P and NPAT or a polynucleotide including a Site II promoter sequence) can also be detected, e.g., using fluorescence energy transfer (FET) (see, for example, Lakowicz et al., U.S. Pat. No. 5,631,169; and Stavrianopoulos et al., U.S. Pat. No. 4,868,103). A fluorophore label is selected such that a first donor molecule's emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the HiNF-P polypeptide to bind to a target molecule can be accomplished using real-time Biomolecular Interaction Analysis (BIA) (e.g., Sjolander and Urbaniczky, 1991, Anal. Chem. 63:2338-2345, Szabo et al., 1995, Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the HiNF-P polypeptide or a test substance is anchored onto a solid phase. HiNF-P polypeptide/test compound complexes anchored on the solid phase can be detected at the end of the reaction. In general, the HiNF-P polypeptide is anchored onto a solid surface, and the test compound (which is not anchored), is labeled, either directly or indirectly, with detectable labels discussed herein.

It can be desirable to immobilize HiNF-P, an anti-HiNF-P antibody or its target molecule to facilitate separation of complexed from non-complexed forms of one or both of the proteins, as well as to accommodate automation of the assay. Binding of a test compound to a HiNF-P polypeptide, or interaction of a HiNF-P polypeptide with a target molecule in the presence and absence of a test or candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/HiNF-P fusion proteins or glutathione-S-transferase/peptidic test compound fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target molecule or HiNF-P polypeptide, and the mixture incubated under conditions conducive for complex formation (e.g., at physiological conditions for salt and pH). Following incubation, the beads or microtiter plate wells are washed to remove any unbound components, the matrix immobilized in the case of beads, and the complex determined either directly or indirectly, for example, as described herein. Alternatively, the complexes can be dissociated from the matrix, and the level of HiNF-P binding or activity determined using standard techniques. Such assays can be used to evaluate the ability of a test compound to modulate the interaction between HiNF-P and a binding partner (e.g., Site II promoter sequence or NPAT).

Other techniques for immobilizing either HiNF-P polypeptide or a target molecule (e.g., a molecule that specifically binds HiNF-P) on matrices include using conjugation of biotin and streptavidin. Biotinylated HiNF-P polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

This assay is performed utilizing antibodies reactive with HiNF-P polypeptide or target molecule, but which do not interfere with binding of the HiNF-P polypeptide to its target molecule. Such antibodies can be bound to the wells of the plate, and unbound target or HiNF-P polypeptide trapped in the wells by antibody conjugation. Methods for detecting such complexes, in addition to those described above for the GST-immobilized complexes, include immunodetection of complexes using antibodies reactive with the HiNF-P polypeptide or target molecule, as well as enzyme-linked assays that rely on detecting an enzymatic activity associated with the HiNF-P polypeptide or target molecule. Test compounds can be added to the assay and their ability to inhibit or enhance HiNF-P binding to a target molecule is assessed, e.g., by comparing the amount of binding in the presence of the test compound with the amount of binding in the absence of the test compound.

Alternatively, cell free assays can be conducted in a liquid phase. In such an assay, the reaction products are separated from unreacted components, by any of a number of standard techniques, including, but not limited to: differential centrifugation (see, for example, Rivas and Minton (1993) Trends Biochem. Sci., 18:284-7); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology, 1999, J. Wiley: New York). Such resins and chromatographic techniques are known to one skilled in the art (see, e.g., Heegaard (1998) J. Mol. Recognit., 11: 141-8; Hage and Tweed (1997) J. Chromatogr. B. Biomed. Sci. Appl., 699:499-525). Fluorescence energy transfer can also be conveniently utilized, as described herein, to detect binding without further purification of the complex from solution. The ability of a test compound to inhibit or increase binding of HiNF-P to a target molecule can be tested using such methods.

The assay can include contacting the HiNF-P polypeptide or biologically active portion thereof with a known compound that binds HiNF-P (e.g., a HiNF-P antibody, NPAT or an HiNF-P-binding fragment thereof, or a polynucleotide comprising a Site II promoter sequence) to form an assay mixture, contacting the assay mixture with a test compound, and determining the ability of the test compound to interact with a HiNF-P polypeptide, as compared to the known compound. The assay can include determining the ability of the test compound to preferentially bind to HiNF-P or biologically active portion thereof, or to modulate the activity of a target molecule, e.g., a Site II promoter sequence or NPAT.

To the extent that HiNF-P can interact in vivo with one or more cellular or extracellular macromolecules, such as proteins, inhibitors of such an interaction are useful. A homogeneous assay can be used to identify inhibitors. For example, a preformed complex of HiNF-P and an interactive cellular or extracellular binding partner (e.g., NPAT or a polynucleotide including a site II promoter sequence) is prepared, such that either the HiNF-P or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt HiNF-P-binding partner interaction can be identified.

Alternatively, HiNF-P polypeptides can be used as a “bait protein” in a two-hybrid assay or three-hybrid assay (see, e.g., U.S. Pat. No. 5,283,317; Zervos et al. (1993) Cell 72:223-232; Madura et al. (1993) J. Biol. Chem., 268:12046-12054; Bartel et al. (1993) Biotechniques,14:920-924; Iwabuchi et al. (1993) Oncogene, 8:1693-1696; and Brent WO94/10300), to identify other proteins, that bind to or interact with HiNF-P (“HiNF-P-binding proteins” or “HiNF-P-bp”) and are involved in HiNF-P activity. Such HiNF-P-bps can be activators or inhibitors of signals by the HiNF-P polypeptides or HiNF-P targets as, for example, downstream elements of a HiNF-P-mediated signaling pathway.

Modulators of HiNF-P expression can also be identified. For example, a cellular or cell-free mixture is contacted with a test compound, and the levels of HiNF-P mRNA or protein are evaluated relative to the level of HiNF-P mRNA or protein in the absence of the test compound. When levels of HiNF-P mRNA or protein are greater in the presence of the test compound than in its absence, the test compound is identified as a stimulator of HiNF-P mRNA or protein expression. Alternatively, when expression of HiNF-P mRNA or protein is less (i.e., statistically significantly less) in the presence of the test compound than in its absence, the test compound is identified as an inhibitor of HiNF-P mRNA or protein expression. The level of HiNF-P mRNA or protein expression can be determined by methods described herein for detecting HiNF-P mRNA or protein.

A modulating agent can be identified using a cell-based or a cell free assay, and the ability of the agent to modulate the activity of a HiNF-P polypeptide can be confirmed in vivo, e.g., in an animal model for monitoring the enhancement or inhibition of H4 histone transcription or G1 to S phase transition.

This invention further pertains to novel agents identified by the above-described screening assays. Accordingly, it is within the scope of this invention to further use a candidate compound identified as described herein (e.g., a HiNF-P modulating agent, an antisense HiNF-P nucleic acid molecule, a HiNF-P-specific antibody, or a HiNF-P-binding partner) in an appropriate animal model (such as those described above) to determine the efficacy, toxicity, side effects, or mechanism of action, of treatment with such an agent. Furthermore, novel agents identified by the above-described screening assays can be formulated into pharmaceutical compositions used for treatments as described herein.

Modulator Peptides

Peptides can be designed for, or isolated based on the ability to enhance or inhibit the activity of HiNF-P. A synthetic peptide is an artificial peptide of about 30 amino acids or less. In some embodiments, the synthetic peptide can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that typically have one disulfide bond, and a loop of about 4 to 12 non-cysteine residues.

Small synthetic peptides offer several advantages. First, the mass per binding site is low, e.g., such low molecular weight peptide domains can show higher binding activity per gram. Second, the possibility of non-specific binding and/or high antigenicity is minimal because there is only a small surface area. Third, small peptides can be engineered to have unique tethering sites such as terminal polylysine segments, e.g., by chemical synthetic methods. Fourth, a constrained polypeptide structure is likely to retain its functionality in a variety of contexts.

Synthetic peptides can be designed to bind to Site II or NPAT and to be effective competitors with HiNF-P for binding to Site II or NPAT. Alternatively, competition assays can be used to identify peptides that can effectively compete for binding of HiNF-P to Site II or NPAT. Such methods are known in the art. In some embodiments, the synthetic peptide inhibits binding of HiNF-P and NPAT. In some embodiments, the synthetic peptide mimics the action of NPAT on HiNF-P, e.g., binds to and activates HiNF-P in a manner similar to NPAT. The ability of such peptides to enhance or inhibit HiNF-P activity can be determined using methods described herein and methods known in the art, e.g., by measurement of cellular proliferation in the presence and absence of synthetic peptides that bind to HiNF-P, NPAT, or Site II, or that affect the interaction of HiNF-P and NPAT or Site II.

Modulation of HiNF-P by RNA Interference (RNAi)

In some embodiments, HiNF-P expression is modulated (e.g., decreased) by contacting a cell with a small interfering RNA (siRNA) that is targeted to a HiNF-P sequence. The introduction of double-stranded RNA into a cell inhibits gene expression in a sequence dependent fashion. Double-stranded short interfering RNAs (siRNAs) mediate gene silencing by targeting for cleavage messenger RNAs (mRNAs) that contain the sequence of one strand of the siRNA. RNAi is mediated by 21 to 25 nucleotide, double-stranded RNA molecules referred to as small interfering RNAs (siRNAs) that are derived by enzymatic cleavage of long, double-stranded RNA in cells (e.g., Hutvagner et al., 2002, Curr. Opin. Genet. Dev.,12:225-32; Hannon, 2002, Nature, 418:244-51). In some embodiments, the siRNA is designed using an algorithm to enhance specificity, e.g., the SMART algorithm (Dharmacon, Lafayette, Colo.).

Methods of designing, synthesizing, and administering siRNAs are known in the art and siRNAs can be obtained from commercial sources (e.g., Dhannacon, Lafayette, Colo.). In some cases, constructs are used to produce HiNF-P sequences. Phage T7 and T3 promoters can be included in expression vectors to enable transcription of individual strands of the HiNF-P gene when T7 or T3 polymerases are provided. Both T7 and T3 promoters can flank the cloned gene so that use of T7 polymerase can drive expression of one strand and T3 can drive expression of the complementary strand.

HiNF-P RNAi constructs can be introduced into cells by standard techniques (e.g., electroporation or liposome fusion) and the decrease in the level of HiNF-P mRNA can be monitored by Northern Blot analysis, the effect on H4 histone transcription (or the transcription of some other reporter gene, e.g., driven by a Site II promoter sequence), or the effect on G1 to S transition.

HiNF-P Antibodies and Uses Thereof

Antibodies directed to a HiNF-P polypeptide can be used to block the polypeptide from binding to the Site II promoter sequence, thereby blocking enhancement of DNA synthesis and reducing cell proliferation.

Anti-HiNF-P antibodies (HiNF-P antibodies) can be used in screening assays, diagnostically, and may be useful in therapeutic applications. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments, which can be generated by treating the antibody with an enzyme such as pepsin.

The antibody can be a polyclonal, monoclonal, recombinant, e.g., a chimeric or humanized, fully human, non-human (e.g., murine), or single chain antibody. In some embodiments, the antibody has effector function and can fix complement. The antibody can be coupled to a toxin or imaging agent.

A full-length HiNF-P polypeptide or antigenic peptide fragment of HiNF-P can be used as an immunogen or can be used to identify anti-HiNF-P antibodies made with other immunogens, e.g., cells, membrane preparations, and the like. The antigenic peptide of HiNF-P should include at least 8 amino acid residues of the amino acid sequence shown in SEQ ID NO:6, and encompasses an epitope of HiNF-P. In general, the antigenic peptide includes at least 10 amino acid residues, for example, at least 15 amino acid residues, at least 20 amino acid residues, or at least 30 amino acid residues of SEQ ID NO:6.

Examples of peptides that are useful for producing HiNF-P antibodies are Peptide 1 (RYESVEKTQQLLRQPQE, SEQ ID NO:11) and Peptide 2 (CEKLQGIAEEPEIQMV, SEQ ID NO:12). Peptide 1 was a portion of HiNF-P that was microsequenced as described herein. Peptide 2 is the predicted C-terminus of HiNF-P based on the cDNA sequence (Genbank accession BC017234).

In general, epitopes encompassed by the antigenic peptide are regions of HiNF-P located on the surface of the protein, e.g., hydrophilic regions, as well as regions with high antigenicity. For example, an Emini surface probability analysis of the human HiNF-P polypeptide sequence can be used to indicate regions that have a particularly high probability of being localized to the surface of the HiNF-P polypeptide and are thus likely to constitute surface residues useful for targeting antibody production.

The anti-HiNF-P antibody can be a single chain antibody. A single-chain antibody (scFV) can be engineered (for example, 1999, Colcher, et al., Ann. N.Y. Acad. Sci., 880:263-80; and Reiter, 1996, Clin. Cancer Res., 2:245-52). The single chain antibody can be dimerized or multimerized to generate multivalent antibodies having specificities for different epitopes of the same target HiNF-P polypeptide.

In some embodiments, a HiNF-P antibody has reduced or no ability to bind an Fc receptor, e.g., it is an isotype, subtype, fragment or other mutant, which does not support binding to an Fc receptor, e.g., it has a mutated or deleted Fc receptor binding region.

An anti-HiNF-P antibody (e.g., monoclonal antibody) can be used to isolate HiNF-P using methods known in the art, such as affinity chromatography or immunoprecipitation. Moreover, an anti-HiNF-P antibody can be used to detect a HiNF-P polypeptide (e.g., in a cellular lysate or cell supernatant) to evaluate the abundance and pattern of expression of the protein. Anti-HiNF-P antibodies can be used diagnostically to monitor HiNF-P protein levels in tissue as part of a clinical testing procedure, e.g., to determine the efficacy of a given treatment regimen. Such antibodies are also useful for monitoring regulation of the cell cycle.

Detection of HiNF-P antibodies can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; examples of bioluminescent materials include luciferase, luciferin, and aequorin, and examples of suitable radioactive material include ¹²⁵I, ¹³¹I, ³⁵S, or ³H.

Transgenic Animals

The invention also includes non-human transgenic animals. Such animals are useful for studying the function and/or activity of a HiNF-P polypeptide and for identifying and/or evaluating test compounds as potential modulators of HiNF-P activity or expression. As used herein, a “transgenic animal” is a non-human animal, e.g., a mammal, such as a rodent (e.g., a rat or mouse), in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion, of endogenous chromosomal DNA, which typically is integrated into or occurs in the genome of one or more of the cells of a transgenic animal. A transgene can direct the expression of an encoded gene product in one or more cell types or tissues of the transgenic animal, whereas other transgenes, e.g., a knockout, reduce expression. Thus, a transgenic animal can be one in which an endogenous HiNF-P gene has been altered by, e.g., by homologous recombination between an endogenous gene and an exogenous DNA molecule introduced into a cell of the animal, e.g., an embryonic cell of the animal, prior to development of the animal. Alternatively, a transgenic animal can be one in which expression of the endogenous HiNF-P gene has been altered by the insertion or rearrangement of a promoter sequence, e.g., to increase expression of the endogenous HiNF-P gene. In some embodiments, the transgenic animal can have more than the normal number of HiNF-P alleles, e.g., more than two copies of the HiNF-P gene (for example, if an additional HiNF-P gene has been added, driven by a HiNF-P or other promoter), thus increasing expression of HiNF-P.

The invention also includes targeting vectors useful in creating transgenic animals, as described herein, e.g., targeting vectors that ablate an allele of the HiNF-P gene. In some embodiments, the vector includes a non-constitutive promoter, e.g., a conditional promoter that allows controlled ablation of the HiNF-P gene. In some embodiments, the targeting vector is a conditional Cre-Lox vector.

Intronic sequences and polyadenylation signals can also be included in the transgene to increase the efficiency of expression of the transgene. A tissue-specific regulatory sequence(s) can be operably linked to a transgene of the invention to direct expression of a HiNF-P polypeptide to particular cells. A transgenic founder animal can be identified based upon the presence of a HiNF-P transgene in its genome and/or expression of HiNF-P mRNA in tissues or cells of the animals. A transgenic founder animal can then be used to breed additional animals carrying the transgene. Moreover, transgenic animals carrying a transgene encoding a HiNF-P polypeptide can further be bred to other transgenic animals carrying other transgenes.

HiNF-P polypeptides can be expressed in transgenic animals or plants, e.g., a nucleic acid encoding the protein or polypeptide can be introduced into the genome of an animal. In some embodiments the nucleic acid is placed under the control of a tissue specific promoter, e.g., a milk or egg specific promoter, and recovered from the milk or eggs produced by the animal. Suitable animals are mice, pigs, cows, goats, and sheep.

Transgenic mice have been generated using a targeting vector that ablates one allele of the HiNF-P gene (see Example 12). This vector was incorporated the construct into mouse embryonic stem (ES) cells by homologous recombination. The ES cells were injected into blastocysts and germline transmission in the mouse has been achieved. Heterozygous HiNF-P null mice can be bred to homozygosity. Because the HiNF-P homozygous knock-out mouse may exhibit very early embryonic lethality, a novel targeting vector that permits conditional ablation of the HiNF-P locus has also been prepared.

Detection of HiNF-P

HiNF-P can be detected in cells and tissues using anti-HiNF-P antibodies and/or nucleic acid probes. HiNF-P mRNA is expressed in proliferating cells and the protein is targeted to specific subnuclear domains. Overexpression of HiNF-P is associated with increased cellular proliferation. Therefore, detection of HiNF-P has diagnostic value in both clinical and academic settings.

In one aspect of the invention, actively proliferating cells are identified by detecting HiNF-P mRNA by PCR or protein levels by antibodies in normal and pathological tissue specimens.

In another aspect, analysis of the subcellular distribution of HiNF-P by fluorescence microscopy can identify sites in the nucleus that support S phase specific gene expression.

In one aspect, the present invention provides a diagnostic method for detecting the presence and/or levels of HiNF-P in vivo (e.g., in vivo imaging in a subject) or in vitro (e.g., in a biological sample, such as tissue, or a biopsy, e.g., a cancerous tissue biopsy). The in vivo method includes: (i) administering a HiNF-P probe (anti-HiNF-P antibody, nucleic acid probe or binding moiety) to a subject; and (ii) detecting the probe in the subject, e.g., the location, levels, and/or binding of the probe to a HiNF-P mRNA or polypeptide. The detecting can include determining location or time of formation of the binding. A change, e.g., a statistically significant change, in the location/binding of the HiNF-P mRNA or polypeptide in the subject relative to a control can be indicative of the presence of HiNF-P in the sample, e.g., the presence of HiNF-P in an abnormal (e.g., pathophysiological) time, place, or level.

The probes can include a label, e.g., a directly or indirectly detectable substance. Suitable detectable substances include various enzymes, prosthetic groups, fluorescent materials, luminescent materials, and radioactive materials, e.g., as known in the art and described herein.

Imaging of HiNF-P

In some embodiments of the invention, a labeled HiNF-P probe such as an antibody, binding moiety, or nucleic acid that specifically binds to HiNF-P, a fragment thereof, or to a HiNF-P nucleic acid (e.g., gene or mRNA) is used for detecting and imaging HiNF-P in vitro or in vivo, as described herein.

In some cases, the HiNF-P probe is radioactively labeled. Procedures for labeling probes such as polypeptides that specifically bind to HiNF-P with the radioactive isotopes (such as ³⁵S, ¹²⁵I, ³²P, ¹³¹I, ¹⁴C, ³H, ¹⁴C, and ³H are useful in vitro) are generally known. For example, tritium labeling procedures are described in U.S. Pat. No. 4,302,438. Iodinating, tritium labeling, and ³⁵S labeling procedures, e.g., as adapted for murine monoclonal antibodies, are described, e.g., by Goding, (Monoclonal Antibodies: Principles and Practice: Production and Application of Monoclonal Antibodies in Cell Biology, Biochemistry, and Immunology, 2nd ed. (London, Academic Press, 1986) pp. 124-126, and the references cited therein. Other procedures for iodinating polypeptides (such as antibodies) are described by Hunter and Greenwood (1962, Nature 144:945), David et al. (1974, Biochemistry, 13:1014-1021), Greenwood et al. (1963, Biochem. J, 89:114-123), Marchalonis (1969, Biochem. J., 113:299-305), and U.S. Pat. Nos. 3,867,517 and 4,376,110. Radiolabel elements that are useful in imaging include ¹²³I, ¹³¹I, ¹¹¹In, and ^{99m}Tc, for example. Procedures for ^{99m}Tc-labeling are described by Rhodes et al., In Burchiel et al., eds., Tumor Imaging: The Radioimmunochemical Detection of Cancer, (New York: Masson, 1982), pp.111-123, and the references cited therein. Procedures suitable for ¹¹¹In labeling antibodies are described by Hnatowich et al. (1983, J. Immul. Methods, 65:147-157), Hnatowich et al. (1984, J. Applied Radiation, 35:554-557), and Buckley et al. (1984, F.E.B.S. Lett. 166:202-204).

In some embodiments, the probe that specifically binds to HiNF-P is directly labeled. Examples of labels useful for diagnostic imaging in accordance with the present invention include radiolabels such as ^{99m}Tc, ¹³¹I, ¹¹¹In, ¹²³I, ¹²⁵I, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The bioconjugate can be labeled with such reagents using methods known in the art (e.g., Wensel and Meares, Radioimmunoimaging and Radioimmunotherapy, (Elsevier, N.Y., 1983); Colcher et al., 1986, Meth. Enzymol, 121:802-816).

In some embodiments of the invention HiNF-P is detected in vivo. The method includes (i) administering to a subject (e.g., an animal being used as a model) a probe that specifically binds to HiNF-P such as an anti-HiNF-P antibody, nucleic acid, or other binding moiety and (ii) exposing the subject to a means for detecting the agent. For example, the subject can be imaged, e.g., by NMR or other tomographic means. In the case of a probe that is not normally taken up by cells, the probe can be delivered using means known in the art such as packaging in lipid vesicles.

In the case of a radiolabeled ligand used to detect HiNF-P in vivo, the ligand is administered to a subject and is detected or “imaged” in vivo using known techniques such as radionuclear scanning using, e.g., a gamma camera or emission tomography. See e.g., A. R. Bradwell et al., “Developments in Antibody Imaging,” (in: Monoclonal Antibodies for Cancer Detection and Therapy, Baldwin et al., eds., pp 65-85, Academic Press, 1985). Alternatively, a positron emission transaxial tomography scanner, such as designated Pet VI located at Brookhaven National Laboratory, can be used where the radiolabel emits positrons (e.g., ¹¹C, ¹⁸P, ¹⁵O, and ¹³N).

MRI Contrast Agents

Magnetic Resonance Imaging (MRI) uses NMR to visualize internal features of living subject, and is useful for localizing a suitably labeled HiNF-P detecting agent. MRI can be used without radioactive tracer compounds. Some MRI techniques are summarized in EP-A-0 502 814. Generally, the differences related to relaxation time constants T1 and T2 of water protons in different environments are used to generate an image. However, these differences can be insufficient to provide sharp high resolution images.

The differences in these relaxation time constants can be enhanced by contrast agents. Examples of such contrast agents include a number of magnetic agents, e.g., paramagnetic agents (which primarily alter T1) and ferromagnetic or superparamagnetic agents (which primarily alter T2 response). Chelating agents (e.g., EDTA, DTPA and NTA chelates) can be used to attach (and reduce toxicity) of some paramagnetic substances (e.g., Fe⁺³, Mn⁺², Gd⁺³).

DNA Binding

Where the labeled HiNF-P probe used for detecting and imaging HiNF-P in vitro or in vivo is a nucleic acid that specifically binds to a HiNF-P nucleic acid (e.g., gene or mRNA), HiNF-P nucleic acids can be detected in a sample using a DNA binding assay such as those known in the art and described herein (see, e.g., van Wijnen et al., 1992, Mol. Cell. Biol. 12, 3273-3287, and van Wijnen, 1991, et al. J. Cell. Biochem. 46, 174-189).

Methods of Treatment

In some embodiments the invention includes both prophylactic and therapeutic methods of treating a subject at risk of (i.e., susceptible to) or having a disorder, associated with aberrant or excessive HiNF-P expression or activity. This includes a subject at risk of or having a disorder characterized by undesirable cellular proliferation such as a cancer.

As used herein, “treatment” means the application or administration of a therapeutic agent to a patient (e.g., a human or a veterinary or experimental animal subject), or application or administration of a therapeutic agent to an isolated tissue or cell line from a patient, who has a disease, a symptom of disease, or a predisposition to get a disease, with the purpose to cure or alleviate or otherwise positively affect the disease, symptoms of disease, or the predisposition to get the disease. A therapeutic agent can include, but is not limited to, small molecules including peptidomimetics, peptoids, nucleic acids (e.g., anti-sense oligonucleotides, and siRNAs), aptamers, carbohydrates, polysaccharides, non-nucleic acid small organic molecules, inorganic molecules, polypeptides, antibodies, ribozymes, and drugs.

In one aspect, the invention includes a method for preventing a disease or condition associated with an aberrant or excessive HiNF-P expression or activity in a subject, by administering to the subject a HiNF-P compound (e.g., polypeptide or polynucleotide), or an agent that modulates HiNF-P expression or at least one HiNF-P activity. Subjects at risk for a disease that is caused or contributed to by aberrant or excessive HiNF-P expression or activity can be identified by, for example, any one or a combination of the assays described herein for measuring HiNF-P expression or activity, or by diagnosing a disorder associated with an abnormal amount of cellular proliferation. Administration of a prophylactic agent can occur prior to the manifestation of symptoms characteristic of the HiNF-P aberrance, such that a disease or disorder is inhibited or, alternatively, delayed in its progression. Depending on the type of HiNF-P aberrance, for example, HiNF-P, a HiNF-P agonist, or a HiNF-P antagonist agent can be used for treating the subject. The appropriate agent can be determined based on screening assays described herein.

Some HiNF-P-related disorders can be caused, at least in part, by an abnormal level of a HiNF-P gene product, or by the presence of a HiNF-P gene product exhibiting abnormal activity. As such, the reduction in the level and/or activity of such gene products would bring about the amelioration of disorder symptoms.

Agents that affect HiNF-P expression or activity can act as therapeutic agents for controlling one or more of cellular proliferative and/or differentiative disorders, disorders associated with bone metabolism, immune disorders, cardiovascular disorders, liver disorders, viral diseases, pain or metabolic disorders. Examples of cellular proliferative and/or differentiative disorders include cancer, e.g., carcinoma, sarcoma, metastatic disorders, or hematopoietic neoplastic disorders, e.g., leukemias. A metastatic tumor can arise from a multitude of primary tumor types, including, but not limited to, those of prostate, colon, lung, breast and liver origin.

As used herein, the terms “cancer,” “hyperproliferative,” and “neoplastic” refer to cells having the capacity for autonomous growth, i.e., an abnormal state or condition characterized by rapidly proliferating cell growth. Hyperproliferative and neoplastic disease states may be categorized as pathologic, i.e, characterizing or constituting a disease state, or may be categorized as non-pathologic, i.e., a deviation from normal, but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas, which include malignancies such as most colon cancers, renal-cell carcinoma, prostate cancer and/or testicular tumors, non-small cell carcinoma of the lung, cancer of the small intestine, and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon, and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is known in the art and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Typically, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, 1991, Crit Rev. in Oncol./Hemotol. 11:267-297). Lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Aberrant expression and/or activity of HiNF-P molecules can mediate disorders associated with bone metabolism. “Bone metabolism” refers to direct or indirect effects in the formation or degeneration of bone structures, e.g., bone formation, or resorption, which can ultimately affect the concentrations in serum of calcium and phosphate. This term also includes activities mediated by HiNF-P in bone cells, e.g., osteoclasts and osteoblasts, that can in turn result in bone formation and degeneration. For example, HiNF-P molecules may support different activities of bone resorbing osteoclasts such as the stimulation of differentiation of monocytes and mononuclear phagocytes into osteoclasts. Accordingly, HiNF-P molecules that modulate the production of bone cells can influence bone formation and degeneration, and thus can be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyroidism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Additionally, HiNF-P molecules may play an important role in the etiology of certain viral diseases, including, but not limited to hepatitis B, hepatitis C and Herpes Simplex Virus (HSV). Modulators of HiNF-P activity can be used to control viral diseases in which the viral genome adversely affects proliferation of a host cell. The modulators can be used in the treatment of viral infected tissue or virus-associated tissue fibrosis, especially liver and liver fibrosis. Also, HiNF-P modulators can be used in the treatment and/or diagnosis of virus-associated carcinoma, for example, hepatocellular cancer.

As discussed, successful treatment of HiNF-P disorders can be brought about by techniques that serve to inhibit the expression or activity of target gene products. For example, compounds, e.g., an agent identified using an assay described herein, that inhibits HiNF-P expression or activity, can be used to prevent and/or ameliorate symptoms of HiNF-P-related disorders. Such molecules can include, but are not limited to polypeptides, phosphopeptides, small non-nucleic acid organic molecules, small inorganic molecules, nucleic acids (including antisense oligonucleotides, siRNAs, and ribozymes), polysaccharides, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of a HiNF-P gene can also be used to reduce the level of target gene expression, thus effectively reducing the level of target gene activity. Triple helix molecules can also be used to reduce the level of target gene activity. Antisense, ribozyme, and triple helix molecules are discussed above.

Another method by which nucleic acid molecules can be used for treating or preventing a disease characterized by HiNF-P expression is through the use of aptamer molecules specific for a HiNF-P polypeptide. Aptamers are nucleic acid molecules having a tertiary structure that permits them to specifically bind to protein ligands (e.g., Osborne et al., 1997, Curr. Opin. Chem Biol., 1:5-9; Patel, 1997, Curr Opin. Chem. Biol., 1:32-46). Since nucleic acid molecules can in many cases be more conveniently introduced into target cells than therapeutic protein molecules, aptamers provide a method by which HiNF-P polypeptide activity can be specifically decreased without the introduction of drugs or other molecules, which may have pluripotent effects.

In circumstances wherein injection of an animal or a human subject with a HiNF-P polypeptide or epitope for stimulating antibody production is harmful to the subject, it is possible to generate an immune response against HiNF-P through the use of anti-idiotypic antibodies (see, for example, Herlyn, D. (1999) Ann. Med., 31:66-78; and Bhattacharya-Chatterjee, M., and Foon, K. A. (1998) Cancer Treat. Res., 94:51-68). If an anti-idiotypic antibody is introduced into a mammal or human subject, it should stimulate the production of anti-anti-idiotypic antibodies, which should be specific to the HiNF-P polypeptide. Vaccines directed to a disease characterized by HiNF-P expression can also be generated in this fashion.

In instances where the target antigen is intracellular and whole antibodies are used, internalizing antibodies can be utilized. Lipofectin™ or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single-chain antibodies can be administered, for example, by expressing nucleotide sequences encoding single-chain antibodies within the target cell population (e.g., Marasco et al., 1993, Proc. Natl. Acad. Sci. USA, 90:7889-7893).

Compounds that inhibit target gene expression, synthesis, and/or activity can be administered to a patient at therapeutically effective doses to prevent, treat, or ameliorate HiNF-P disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosages for use in humans. The dosage of such compounds typically lies within a range of circulating concentrations that include the ED₅₀ with little or no toxicity. The dosage can-vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography. Another way to determine an effective dose for an individual is to assay the “free” and “bound” compound levels in the serum of the test subject. Such assays can utilize antibody mimics and/or “biosensors” that have been created through molecular imprinting techniques. A compound that can modulate HiNF-P activity is used as a template, or “imprinting molecule,” to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell, R. J. et al (1996) Current Opinion in Biotechnology, 7:89-94 and in Shea, K. J. (1994) Trends in Polymer Science, 2:166-173. Such “imprinted” affinity matrixes are amenable to ligand-binding assays, whereby the immobilized monoclonal antibody component is replaced by an appropriately imprinted matrix. An example of the use of such matrixes can be seen in Vlatakis, G. et al (1993) Nature, 361:645-647. Through the use of isotope labeling, the “free” concentration of compound that modulates the expression or activity of HiNF-P can be readily monitored and used in calculations of IC₅₀.

Such “imprinted” affinity matrixes can also be designed to include fluorescent groups whose photon-emitting properties measurably change upon local and selective binding to a target compound. These changes can be readily assayed in real time using appropriate fiber optic devices, in turn allowing the dose in a test subject to be quickly optimized based on a compound's individual IC₅₀. A rudimentary example of such a “biosensor” is discussed in Kriz, D. et al (1995) Analytical Chemistry, 67:2142-2144.

To modulate HiNF-P expression or activity for therapeutic purposes, a cell is contacted with a HiNF-P or agent that modulates one or more of the activities of HiNF-P polypeptide activity associated with the cell. An agent that modulates HiNF-P polypeptide activity can be an agent as described herein, such as a nucleic acid or a protein, a naturally-occurring target molecule of a HiNF-P polypeptide (e.g., a HiNF-P substrate or receptor), a HiNF-P antibody, a HiNF-P agonist or antagonist, a peptidomimetic of a HiNF-P agonist or antagonist, or other small molecule.

A HiNF-P modulating agent can, in some embodiments, stimulate one or more HiNF-P activities. Examples of such stimulatory agents include active HiNF-P polypeptide and a nucleic acid molecule encoding HiNF-P polypeptide. In another embodiment, the agent inhibits one or more HiNF-P activities. Examples of such inhibitory agents include antisense HiNF-P nucleic acid molecules, anti-HiNF-P antibodies, and HiNF-P inhibitors. These modulatory methods can be performed in vitro (e.g., by culturing the cell with the agent) or, alternatively, in vivo (e.g., by administering the agent to a subject). Thus, an individual afflicted with a disease or disorder characterized by aberrant or excessive expression or activity of a HiNF-P polypeptide or nucleic acid molecule can be treated using a HiNF-P agent. The method of treatment can involve administering an agent (e.g., an agent identified by a screening assay described herein), or combination of agents that modulates (e.g., up regulates or down regulates) HiNF-P expression or activity. In another embodiment, the method involves administering a HiNF-P polypeptide or nucleic acid molecule as therapy to compensate for reduced, aberrant, or excessive HiNF-P expression or activity.

Stimulation of HiNF-P activity is desirable in situations in which HiNF-P is abnormally downregulated and/or in which increased HiNF-P activity is likely to have a beneficial effect. Likewise, inhibition of HiNF-P activity is desirable in situations in which HiNF-P is abnormally upregulated and/or in which decreased HiNF-P activity is likely to have a beneficial effect.

Pharmaceutical Compositions

In another aspect, the present invention provides compositions, e.g., pharmaceutically acceptable compositions, which include agents that can modulate HiNF-P expression or activity, e.g., an anti-HiNF-P antibody or antisense HiNF-P oligonucleotide, formulated together with a pharmaceutically acceptable carrier. As used herein, pharmaceutical compositions” encompass diagnostic compositions (e.g., for in vivo imaging) as well as therapeutic compositions.

As used herein, the term “pharmaceutically acceptable carrier” includes any and all solvents, dispersion media, coatings, antibacterial and antifungal agents, isotonic and absorption delaying agents, and the like that are physiologically compatible. In general, these carriers are suitable for intravenous, intramuscular, subcutaneous, parenteral, spinal, or epidermal administration (e.g., by injection or infusion). A “pharmaceutically acceptable salt” refers to a salt that retains the desired biological activity of the parent compound and does not impart any undesired toxicological effects (see e.g., Berge et al., 1977, J. Pharm. Sci., 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkali metals sodium and potassium, alkaline earths, such as magnesium, calcium and the like, as well as from nontoxic organic amines, such as N,N′-dibenzylethylenediamine, N-methylglucamine, chloroprocaine, choline, diethanolamine, ethylenediamine, procaine and the like.

Also, if the compound is a polypeptide encoded by DNA, the compound can be used for gene therapy by integrating the DNA into a vector for gene therapy. Although the dosage amount and method of administration differ according to the body-weight, age, and symptoms of a patient, one skilled in the art can suitably select these.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection, and infusion.

Pharmaceutical compositions typically are sterile and stable under the conditions of manufacture and storage. A pharmaceutical composition can also be tested to insure it meets regulatory and industry standards for administration. For example, endotoxin levels of the preparation can be tested using the Limulus amebocyte lysate assay (e.g., using the kit from Bio Whittaker lot # 7L3790, sensitivity 0.125 EU/ml) according to the United States Pharmacopeia (USP 24/NF 19) methods. Sterility of pharmaceutical compositions can be determined using thioglycolate medium according to the United States Pharmacopeia (USP 24/NF 19) methods. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the bioconjugate) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, useful methods of preparation are vacuum drying and freeze-drying that yield a powder of the active ingredient plus any additional desired ingredient from a previously sterile-filtered solution.

In certain embodiments, a bioconjugate is orally administered (for example, when a tumor is accessible from the lumen of the gut), for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the compounds can be incorporated with excipients and used in the form of ingestible tablets, buccal tablets, troches, capsules, elixirs, suspensions, syrups, wafers, and the like. To administer a compound of the invention by other than parenteral administration, it can be necessary to coat the compound with, or co-administer the compound with, a material to prevent its inactivation.

Pharmaceutical compositions can be administered with medical devices known in the art. For example, in one embodiment, a pharmaceutical composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

Systemic administration of a HiNF-P composition described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal or topical administration, the active compounds are typically formulated into ointments, salves, gels, or creams as generally known in the art. Such methods of administration would be particularly useful for treating wounds, e.g., increasing cell proliferation (e.g., of skin cells) to aid in wound healing.

In certain embodiments, the compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes can comprise one or more moieties that are selectively transported into specific cells or organs, thus enhance targeted drug delivery (e.g., Ranade, 1989, J. Clin. Pharmacol. 29:685).

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The pharmaceutical compositions can include a “therapeutically effective amount” or a “prophylactically effective amount” of a composition. A “therapeutically effective amount” refers to an amount effective, at dosages and for periods of time necessary, to achieve the desired clinical treatment result. A therapeutically effective amount of the composition can vary according to factors such as the disease state, age, sex, and weight of the individual, and the ability of the protein ligand to elicit a desired response in the individual. A therapeutically effective amount is also one in which any toxic or detrimental effect of the composition is outweighed by the therapeutically beneficial effects. A “therapeutically effective dosage” inhibits a measurable clinical parameter, e.g., tumor growth rate by at least about 20%. In some embodiments, tumor growth is inhibited by at least about 40% or by at least about 75%, and typically causes the tumor to shrink relative to untreated subjects. The ability of a compound to inhibit a measurable parameter, e.g., tumor growth rate, can be evaluated in an animal model system predictive of efficacy in human tumors. Alternatively, this property of a composition can be evaluated by examining the ability of the compound to inhibit, such inhibition in vitro by assays known to the skilled e in which any toxic or detrimental effect of the composition is outweighed by the

A “prophylactically effective amount” refers to an amount effective, at dosages and for periods of time necessary, to achieve a desired clinical preventative result. Typically, since a prophylactic dose is used in subjects prior to or at an earlier stage of disease to prevent development or progression of the disease, the prophylactically effective amount will be less than the therapeutically effective amount.

The present invention encompasses agents that modulate expression or activity of HinF-P. An agent may, for example, be a small molecule. For example, such small molecules include, but are not limited to, peptides, peptidomimetics (e.g., peptoids), amino acids, amino acid analogs, polynucleotides (e.g., antisense oligonucleotides or siRNAs), polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Also within the scope of the invention are kits that include a composition of the invention and instructions for use, e.g., treatment, prophylactic, or diagnostic use. In one embodiment, the instructions for diagnostic applications include the use of a bioconjugate having a targeting moiety that recognizes a cancer-specific antigen to detect a cancer cell, in vitro, e.g., in a sample, e.g., a biopsy or cells from a patient having a cancer or neoplastic disorder, or in vivo. In another embodiment, the instructions for therapeutic applications include suggested dosages and/or modes of administration in a patient with a cancer or neoplastic disorder.

A therapeutically effective amount of protein or polypeptide (i.e., an effective dosage) ranges from about 0.001 to 30 mg/kg body weight, for example, about 0.01 to 25 mg/kg body weight, about 0.1 to 20 mg/kg body weight, and in some cases about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, for example, between 2 to 8 weeks, between about 3 to 7 weeks, and in some cases for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, can include a series of treatments.

For antibodies, the dosage can be, e.g., 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology, 14:193).

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

The invention is further described in the following examples, which do not limit the scope of the invention described in the claims.

### EXAMPLES

### Example 1

### Isolation and Characterization of HiNF-P

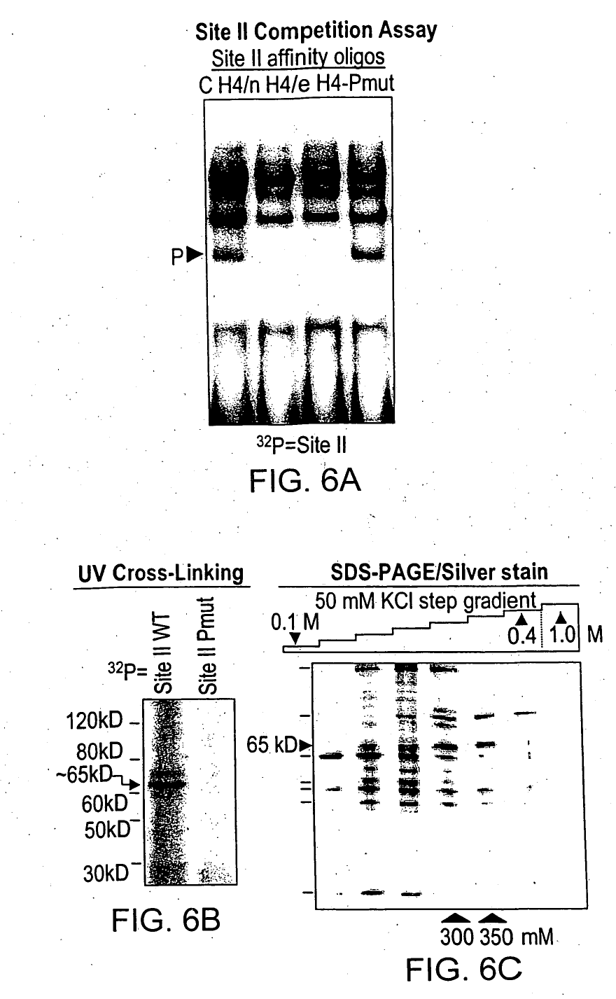

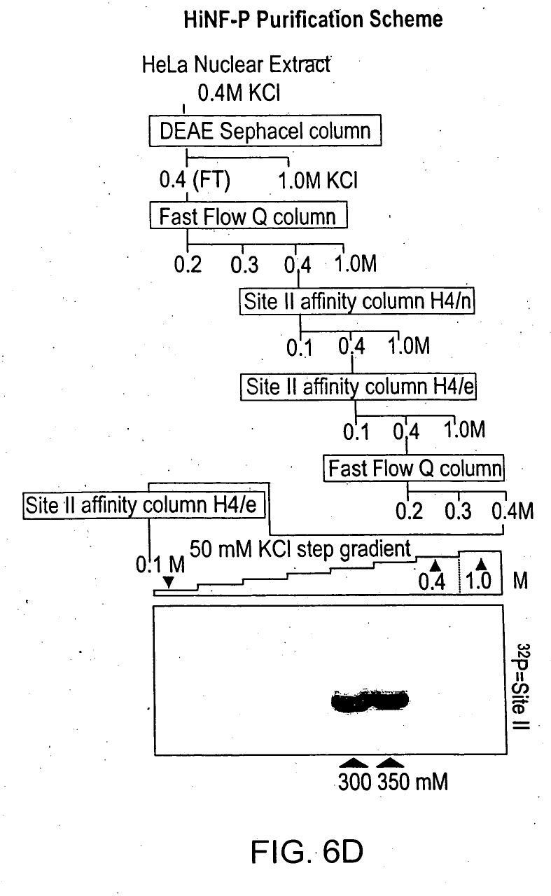

To identify the protein associated with histone H4 gene activation and HiNF-P activity the protocol outlined in FIG. 6D was used. Biochemical purification of HiNF-P was performed using two distinct sequence-specific affinity resins that are based on known high affinity binding sites for HiNF-P in the H4/n and H4/e genes (FIG. 6A). The presence of HiNF-P was monitored by electrophoretic mobility shift assays (EMSA).

EMSA competition assays were performed to test binding of HiNF-P to two different oligonucleotides that were used to make DNA affinity columns. HiNF-P in HeLa nuclear extracts binds to the ³²P labeled minimal binding sequence (5′-GATCCTTCGGTTTTCAATCTGGTCCGAT-3′, SEQ ID NO:1 (FIG. 6A, Lane C)). HiNF-P binding is totally abolished when cold excess double-stranded oligo is present (FIG. 6A, Lanes H4/n and H4/e), but binding is unaffected by addition of excess mutant Site II oligonucleotide (5′-GATCCTTCGGTTTTCAATCTTCTACGAT-3′, SEQ ID NO:2 (FIG. 6A, Lane H4-Pmut).

Preparation of Nuclear Extracts Containing HiNF-P

HiNF-P protein was isolated from HeLaS3 cells (National Cell Culture Center). Exponentially growing cells were harvested at 0.6×10⁶/ml (total volume 160 L) after culturing in Joklik's MEM with 5% newborn calf serum (NCS), 1% non-essential amino acids (NEAA), 4 mM L-glutamine, 2.0 g/l NaHCO₃, and 1% penicillin/streptomycin. Cells were harvested by centrifugation and pellets containing 10×10⁹ cells each were quickly frozen in liquid nitrogen. Nuclear extracts were prepared essentially as described in van Wijnen et al. (J. Cell. Biochem., 46(2):174-89, 1991) and van Wijnen et al. (Mol. Cell. Biol., 12:3273-3287, 1992). Briefly, cells were homogenized successively in buffer T (10 mM HEPES/NaOH, pH 7.5, 10 mM KCl, 1 mM EDTA, 300 mM sucrose), buffer A (same as buffer T without sucrose), and KN400 buffer (20 mM HEPES/NaOH, pH 7.5, 20% glycerol, 400 mM KCl, 0.2 mM EDTA, 1 mM EGTA (pH 8.0) and 0.01% NP-40). All extraction buffers contained COMPLETE protease inhibitor cocktail (Roche).

Fractionation of Nuclear Extracts

Nuclear extracts were fractionated by a combination of ion-exchange and DNA affinity chromatography. Chromatographic fractions were analyzed for HiNF-P activity by electrophoretic mobility shift assays (EMSAs).

To purify HiNF-P, nuclear proteins were first passed through a DEAE Sephacel® column primed with KN400 buffer. The flow through containing HiNF-P DNA binding activity was diluted with an equal volume of the same buffer without KCl (KNO) and loaded onto a Fast Q-Sepharose column. The column was washed with KN300, KN400 and KN1000. The KN400 fraction, which contains HiNF-P DNA binding activity, was mixed with three volumes of KNO containing 6400 μg of salmon sperm DNA, 1 mM dithiothreitol (DTT), 20 mM MgCl₂, and 10 mM ZnCl₂. The mixture was then passed through a DNA affinity column containing a multimerized oligonucleotide spanning Site II in the H4/n gene and washed with KN400 and KN1000. The KN400 fraction was collected and diluted four-fold with KN0 containing 640 μg salmon sperm DNA, 1 mM DTT, 20 mM MgCl₂, 10 mM ZnCl₂. This fraction was then loaded on a DNA affinity column using a multimerized oligonucleotide spanning the HiNF-P site in the H4/e gene. The H4/e Site II column was washed with KN400 and KN1000 and the KN400 fraction containing HiNF-P was concentrated by passage through a Fast Q-Sepharose column (KN400 fraction collected). This fraction was reapplied to the H4/e Site II affinity column in the presence of 10 μg of a Site II oligonucleotide with a mutation in the HiNF-P binding site. Final fractions were eluted from the H4/e Site II affinity resin using a 50 mM KCl step gradient and all fractions were analyzed for HiNF-P DNA binding activity. Only KN300 (300 mM KCl) and KN350 (350 mM KCl) fractions were found to contain HiNF-P DNA binding activity (FIG. 6B, bottom panel).

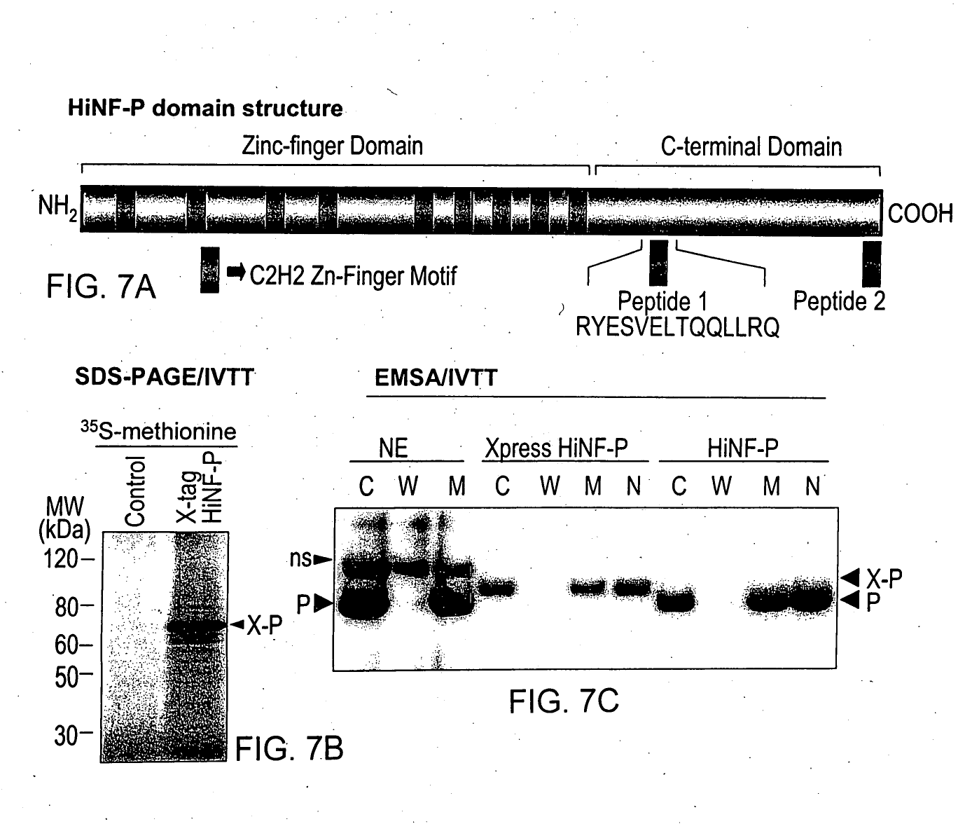

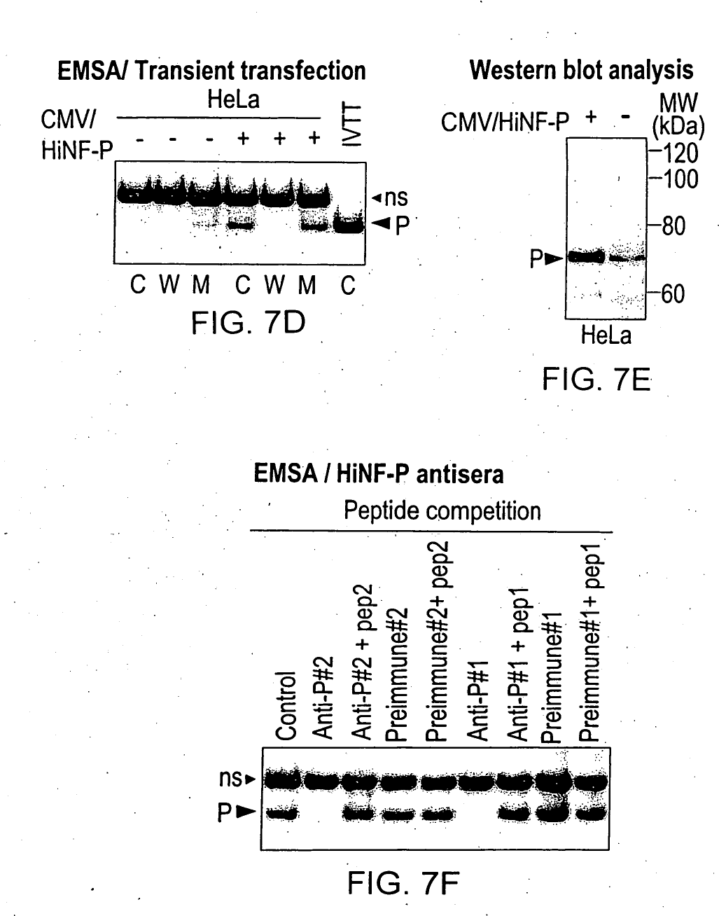

Proteins present in each of the fractions were analyzed in 10% SDS-PAGE gels followed by silver staining. A prominent 65 kDa band present only in the 300 and 350 mM KCl fractions was analyzed by Matrix-Assisted-Laser-Desorption-Ionization Time-of-Flight (MALDITOF)/mass spectrometry and peptide microsequencing. Peptide microsequencing indicated that the 65 kDa protein contains the amino acids RYESVELTQQLLRQPQE (Peptide 1; SEQ ID NO:11; FIG. 7A). The 14 amino acid sequence of Peptide 1 was reproducibly detected in three HiNF-P preparations from 2×10¹⁰ HeLa cells each.

Electrophoretic Mobility Shift Assays

Electrophoretic mobility shift assays (EMSAs) used to identify HiNF-P were performed using the method described by van Wijnen et al. (1992, Mol. Cell. Biol., 12:3273-3287), and van Wijnen et al. (1991, J. Cell. Biochem., 46:174-189). Protein mixtures (10 μl) were prepared by adding either partially purified fraction or in vitro synthesized recombinant HiNF-P in KN100 buffer containing 0.2 mM MgCl₂ and 0.2 mM ZnCl₂. DNA mixtures (10 μl) containing 10 fmol DNA probe, 0.2 μg salmon sperm DNA (Sigma), 1 mM dithiothreitol (DTT), 0.2 mM MgCl₂, and 0.2 mM ZnCl₂ were combined with the protein mixtures and incubated for 20 minutes at room temperature prior to electrophoresis in a 4% nondenaturing polyacrylamide gel. For competition experiments, 1 pmol unlabeled double-stranded oligonucleotide was added in a total reaction mixture of 20 μl. For immuno-EMSAs, antisera against Peptide 1 or Peptide 2 were added to the binding reaction. As controls, the antisera were neutralized by incubation with excess amounts of the matching peptides before addition to the binding mixtures.

EMSA can be used to detect the presence of HiNF-P polypeptide as well as screening for compounds that can block the binding of HiNF-P to Site II. Such compounds are candidates for inhibiting histone H4 activation and inhibiting cell proliferation.

### Example 2

### Molecular Weight Determination

To determine the molecular weight of HiNF-P, ultraviolet cross-linking of HiNF-P from a nuclear extract to a BrdU- and ³²P-labeled probe spanning Site II sequences was used.

Ultraviolet cross-linking experiments were performed by adapting a method described in Ausubel, F. M. et al. (Current Protocols in Molecular Biology. Greene and Wiley, New York, 1989). A radiolabeled and BrdU-substituted probe was prepared by incubating 20 pmol of partially double-stranded HiNF-P-specific oligonucleotide (top strand: 5′-CGC TTT CGG-3′, SEQ ID NO:13; Bottom strand: 5′-CAA GAG TAT CGG ACC AGA TTG AAA ACC GAA AGC G-3′, SEQ ID NO:14) with 1 μl of 50× dNTP/BrdUTP solution (containing 50 μM DATP, 50 μM dGTP, 37.5 μM dTTP and 12.5 μM BrdUTP), 150 μCi α-³²P-dCTP and 1 μl of a solution containing 5 mM DTT and 1 μg/μl BSA, in a total reaction volume of 25 μl. Labeling was initiated by the addition of 10 units Klenow fragment (New England Biolabs) and the mixture incubated for 90 minutes at 16° C. The reaction was completed by adding 5 μl of a chase solution containing 0.6 μM dCTP at 16° C. for 30 minutes. Each BrdU-substituted probe was then purified through a Sephadex® G-50 column. Protein-DNA binding reactions were performed as described for EMSAs using a chromatographic fraction enriched for HiNF-P. A Fast Flow Q fraction (7.5 μl; 0.4 M KCl) was incubated in a 50 μl reaction mixture containing 10 fmol of BrdU substituted probe, 1 mM DTT and 100 μM each of Zn²⁺ and Mg²⁺. Cross-linking was carried out using an inverted UV transilluminator (305 nm). Following nuclease digestion, the products were analyzed by 10% SDS-PAGE.

These experiments revealed that HiNF-P is a 65 kDa protein (FIG. 6C). Consistent with this result, chromatographic fractions containing maximal HiNF-P DNA binding activity in gel shift assays (FIG. 6B) also exhibited significant and reproducible enrichment of a 65 kDa protein in silver-stained SDS/PAGE gels (FIG. 6D).

### Example 3

### Cloning of HiNF-P

To identify a nucleic acid sequence that encodes HiNF-P, a database was scanned for sequences that encode Peptide 1. Peptide 1 was identified within a 2.1 kb cDNA (Genbank Accession BC017234) with an open reading frame that encodes a Zn finger protein with a predicted size of 517 amino acids (FIG. 7A). Specific primers (Forward: 5′-TCA GGG ATC CGT CCG CCT CCT GGG AAA GTT CC-3′, SEQ ID NO:15; Reverse: 5′-TCC CTC GAG CAA CCA TCT GGA TCT CTG GCT CCT C-3′, SEQ ID NO:16) were used to amplify a approximately 1.5 kb DNA segment spanning the entire open reading frame by RT-PCR using total HeLa mRNA. The RT-PCR product was cloned as a BamHI-XhoI fragment into pCDNA3.1/A (Invitrogen, Calif.) for expression in eukaryotic cells. In vitro transcription and translation (IVTT) reactions were performed by incubating plasmid DNA (1.2 μg) with 25 μl of rabbit reticulocyte lysate, 30 units of RNasin, 1 μl of T7 RNA polymerase (Promega) and 1 μl of 1 mM L-methionine or 45 μCi of [³⁵S-] methionine (1000 Ci/mmol; New England Nuclear) in a reaction mixture with a total volume of 60 μl at 300C for 2-3 hours. Synthesis was stopped by adding 20 μl of 80% glycerol and an aliquot was analyzed by 10% SDS-PAGE to confirm synthesis of the full-length HiNF-P protein.

The recombinant protein translated in vitro from this cDNA migrates with the expected molecular mass based on SDS-PAGE (FIG. 7B). The recombinant protein, with or without an epitope tag, interacts specifically with Site II and exhibits oligonucleotide competition characteristics in EMSAs that are indistinguishable from endogenous HiNF-P isolated from HeLa cells (FIGS. 7C and 7D). In FIG. 7C, the SPI oligonucleotide is a control non-specific oligonucleotide that includes a binding site for the general transcription factor SPI. The DNA bound protein without the epitope-tag co-migrates with the HeLa HiNF-P/Site II complex (FIG. 7C). Furthermore, expression of the recombinant protein in HeLa cells increases the amount of HiNF-P DNA binding activity (FIG. 7D). Thus, a sequence-specific DNA binding protein with the biochemical properties of the histone H4 gene-specific regulatory factor HiNF-P was purified and a clone encoding the protein was identified.

The identified sequences are useful for, e.g., generating antibodies that specifically bind HiNF-P and screening assays for compounds that inhibit HiNF-P expression or activity.

### Example 4

### HiNF-P Antibodies

To establish that the recombinant protein described above is HiNF-P, antibodies were prepared against two different HiNF-P peptides. One antibody was directed against the segment of the protein spanning Peptide 1 (SEQ ID NO:11), which was identified by microsequencing. The second antibody was raised against the predicted C-terminal sequence (Peptide 2; SEQ ID NO:12) of the protein (see FIG. 6A). Antibodies against the peptides were raised in rabbits (ResGen, Invitrogen Corporation, Huntsville, Ala.). Antibodies were used as antiserum, IgG fraction, or peptide affinity purified fraction for Western blots, EMSAs, immunoprecipitations, and immunofluorescence microscopy.

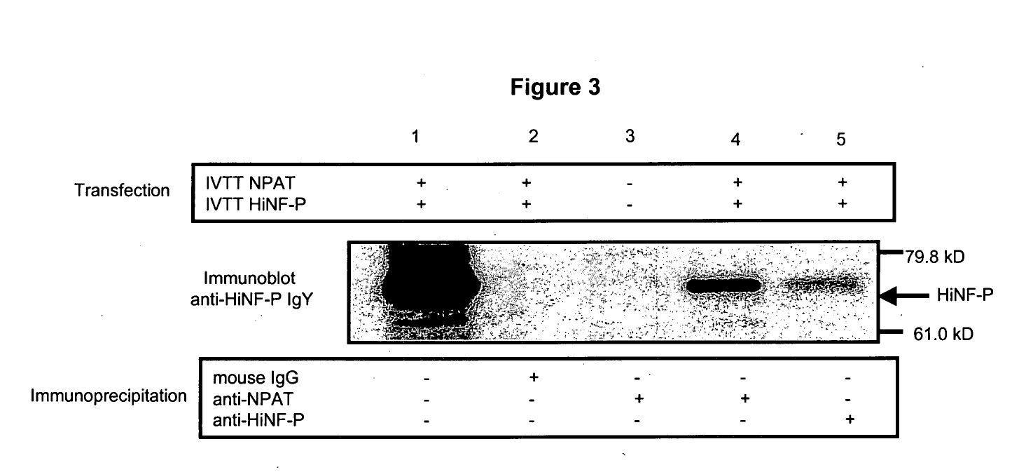

Western blot analysis revealed that the antibodies detect both endogenous and recombinant HiNF-P as proteins with the same apparent molecular mass (FIG. 3E). Both of the antisera containing HiNF-P antibodies that were used in the experiments can disrupt formation of the endogenous HiNF-P complex in gel shift assays, but pre-incubation of the antisera with the corresponding antigenic peptide prevents this disruption (FIG. 7G).

### Example 5

### Localization of HiNF-P

Immunofluorescence microscopy was used to detect the subcellular localization of HiNF-P. Localization was done in whole cells, cytoskeletal preparations, and nuclear matrix intermediate filament preparations. Generally, human osteosarcoma SAOS-2 cells were grown on cover slips (Fisher Scientific, Springfield, N.J.) and extracted according to procedures documented in Choi et al. (1998, J. Cell Sci., 3035-3043), Tang et al. (1999, Biol. Chem., 274, 33580-33586) and Zeng et al. (1998, Proc. Natl. Acad. Sci. USA, 95, 1585-1589).

For whole cell preparations, cells were rinsed twice with ice cold PBS and fixed with 3.7% formaldehyde in PBS for 10 minutes on ice. After rinsing twice with PBS, the cells were permeabilized in 0.1% Triton X-100 in PBS, and rinsed twice with PBSA (0.5% bovine serum albumin (BSA) in phosphate buffered saline (PBS)) followed by antibody staining.

For cytoskeletal (CSK) preparations, cells were extracted in ice cold CSK buffer (100 mM NaCl, 0.3 M sucrose, 10 mM PIPES, 3 mM MgCl₂, 1 mM EGTA, 0.5% Triton X-100, pH 6.8) containing RNase inhibitors (2 mM vanadyl ribonucleoside complex (VRC) or 40 U/ml RNasin) twice for 15 minutes each to remove cytosol, followed by 10 minute fixation in 3.7% formaldehyde in CSK buffer (without VRC). Cells were washed in PBSA before antibody staining.

For nuclear matrix intermediate filament (NMIF) preparations, cells were extracted twice for 15 minutes each with ice-cold CSK buffer and then chromatin was digested by two consecutive 30 minutes incubations at 25° C. with the addition of digestion buffer (DB, same as CSK buffer except the NaCl concentration is 50 mM rather than 100 mM) containing 50 U/ml RNase-free DNaseI (Roche Molecular Biochemicals, Indianapolis, Ind.). NMIF was obtained by further extraction for 10 minutes on ice with a stop solution (digestion buffer supplemented with 0.25 M ammonium sulphate). Cells were then fixed in ice-cold digestion buffer containing 3.7% formaldehyde and subjected to antibody staining.

Antibody staining was performed by incubating a whole cell, CSK, or NMIF preparation with rabbit polyclonal HiNF-P antibody for 1 hour at 37° C. Coverslips were rinsed four times with PBSA before addition of a secondary antibody. Secondary antibody was Alexa 488 goat anti-rabbit (Molecular Probes, Eugene, Oreg.). Cells were incubated with 1:800 dilution of the secondary antibody at 37° C. for one hour and then were washed four times with PBSA. The cells were then stained with 4′, 6-diamidino-2-phenylindole DAPI (5 μg/μl) for 5 minutes and washed once with PBSAT (0.1% Triton in PBSA) and twice with PBS. Immunostaining of cell preparations was recorded using an epifluorescence microscope attached to a CCD-camera. The collected digital images were analyzed with the Metamorph software programs.

For example, human osteosarcoma SAOS-2 cells (0.4×10⁶ cells/well) were grown on gelatin-coated cover slips and analyzed by in situ immunofluorescence microscopy as whole cell (WC) and NMIF preparations. HiNF-P was detected using the IgG-purified HiNF-P antibody (α802k). A polyclonal goat anti-rabbit antibody conjugated with FITC (Alexa 488) was used as the secondary antibody. The fluorescent signals were visualized with a Zeiss Axioplan microscope equipped with a CCD camera, and MetaMorph Imaging Software was used for image acquisition.

These experiments revealed that both the recombinant and endogenous HiNF-P proteins are localized in the nucleus and are stably associated with distinct subnuclear foci. As shown by the experiments localizing HiNF-P in NIMF, localization was retained even upon removal of chromatin. (FIG. 7F).

### Example 6

### Chromatin Immunoprecipitation Assays

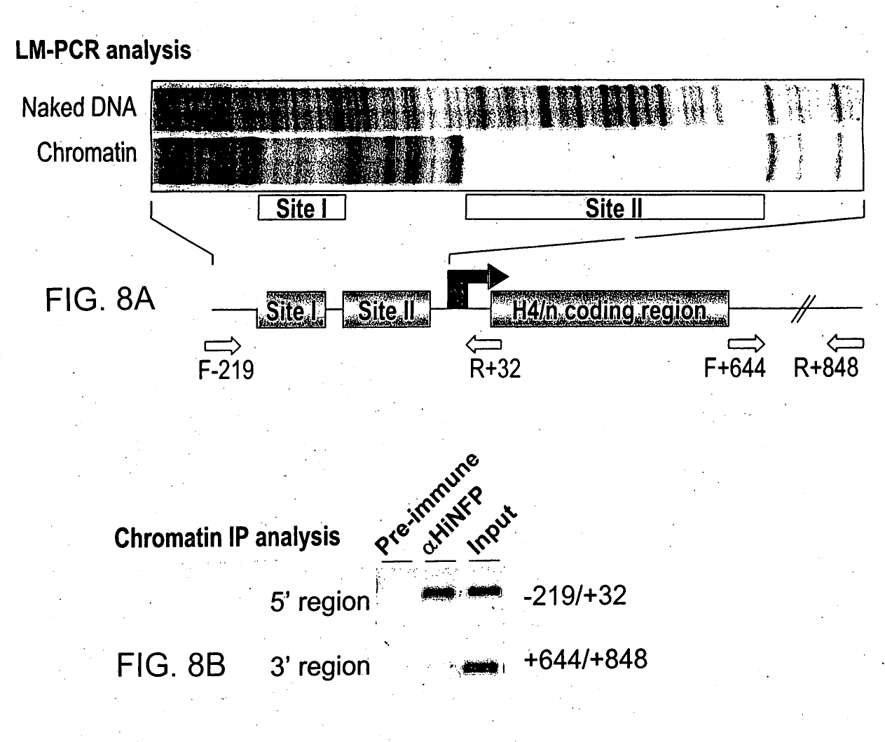

To assess whether HiNF-P interacts with the Site II region of the H4 locus in vivo, chromatin immunoprecipitation assays and genomic footprinting were performed using ligation-mediated PCR (FIGS. 8A and 8B).

In these experiments, the genomic occupancy of the binding element of HiNF-P with Site II was established using ligation-mediated (LM)-PCR assisted DNaseI footprinting in proliferating HL-60 cells. In this procedure, primers were used that amplify the proximal promoter of the histone H4 gene.

Chromatin spanning the H4 promoter was found to contain the expected two regions of DNase I protection that represent protein/DNA interaction domains referred to as Sites I and II (FIG. 8A). The locations and sizes of Site I and II are similar to those previously established in HeLa cells by genomic Southern blotting. Site II is the HiNF-P interaction site (see FIGS. 5A-B). Chromatin immunoprecipitates obtained with the HiNF-P antibody were found to be enriched for a DNA segment that encompasses the HiNF-P binding element within Site II in the H4 promoter, but not for a DNA segment derived from the 3′ non-coding region of the H4 gene that lacks a HiNF-P binding site (FIG. 8B). This provides additional evidence that the protein identified as HiNF-P is indeed that protein. Because the Site II regulatory sequences are highly conserved between species, it is also likely that the HiNF-P protein that binds to this site is also highly conserved.

### Example 7

### Coupling of HiNF-P, Histone H4 Gene Expression, and Cell Proliferation

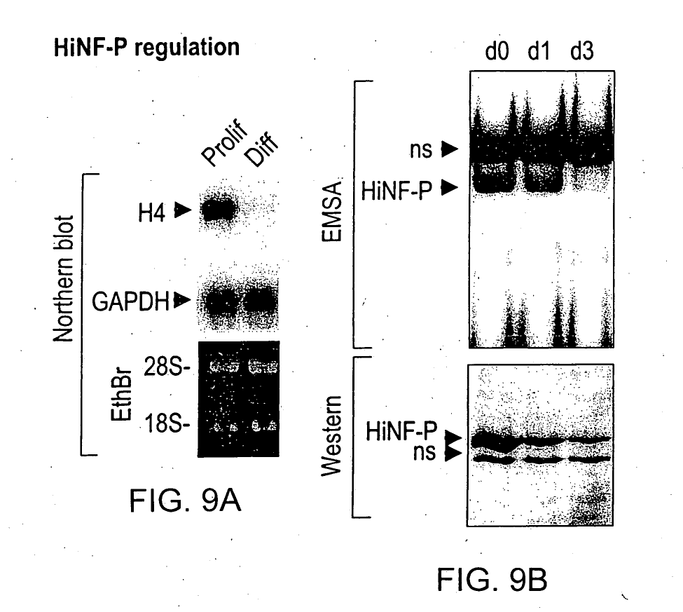

The biological coupling of HiNF-P, histone H4 gene expression and cell proliferation was examined (FIGS. 9A-B). Briefly, human HL-60 promyelocytic leukemia cells were treated with 16 nM PMA (phorbol-12-myristate-13-acetate) to induce differentiation, histone H4 expression and HiNF-P expression were then analyzed. Northern blot analysis of total RNA isolated from proliferating (Prolif) and differentiated (Diff) cells showed that, as expected, histone H4 expression is down-regulated during HL-60 differentiation (FIG. 9A, top). GAPDH mRNA levels are shown in FIG. 9A for comparison. The bottom of FIG. 9A shows the same gel in the region containing 28S and 18S ribosomal RNAs and stained with EtBr to demonstrate equal loading of RNA.

HiNF-P DNA binding activity was assayed using EMSA of nuclear extracts that were prepared from proliferating (d0) HL-60 cells and cells at various times after induction of differentiation with PMA (FIG. 9B, right panel, top; d1=1 day; d3=3 days). Protein levels of HiNF-P in the cells were assayed by Western blot (FIG. 9B, right panel, bottom). The DNA binding activity of HiNF-P was detected in proliferating HL-60 cells that actively transcribe H4 mRNAs, but any binding was below the level of detection in post-proliferative differentiated HL-60 cells that had ceased histone H4 gene expression (FIG. 9B).

Western blot analysis with a HiNF-P-specific antibody showed that HiNF-P protein levels are also regulated with respect to the proliferative status of HL-60 cells. Thus, HiNF-P DNA binding activity and protein levels are coupled to histone H4 gene expression.

### Example 8

### HiNF-P Activates DNA Replication-Dependent Histone H4 Gene Transcription, and NPAT is a HiNF-P-Dependent Transcriptional Co-activator of Cell Cycle Controlled Histone H4 Genes, and Interacts Directly with HiNF-P

The functional contribution of HiNF-P to transcription of the DNA replication dependent histone H4 gene family was examined. As described above, the native H4/n promoter interacts with multiple transcription factors, one of which is HiNF-P. The promoter contains a single HiNF-P binding site that corresponds to the H4 subtype specific consensus element (see FIG. 5A). NPAT, a substrate of the CDK2/cyclin E kinase, enhances histone gene transcription. However, NPAT does not directly bind DNA and the downstream effector through which NPAT regulates histone H4 genes has not been characterized. Thus, a fundamental question has been the functional link between the NPAT/cyclin E/CDK2 signaling pathway and transcriptional control of histone genes at the G1/S phase cell cycle transition. Transient transfection assays were used to investigate the role of NPAT in histone H4 promoter activity.

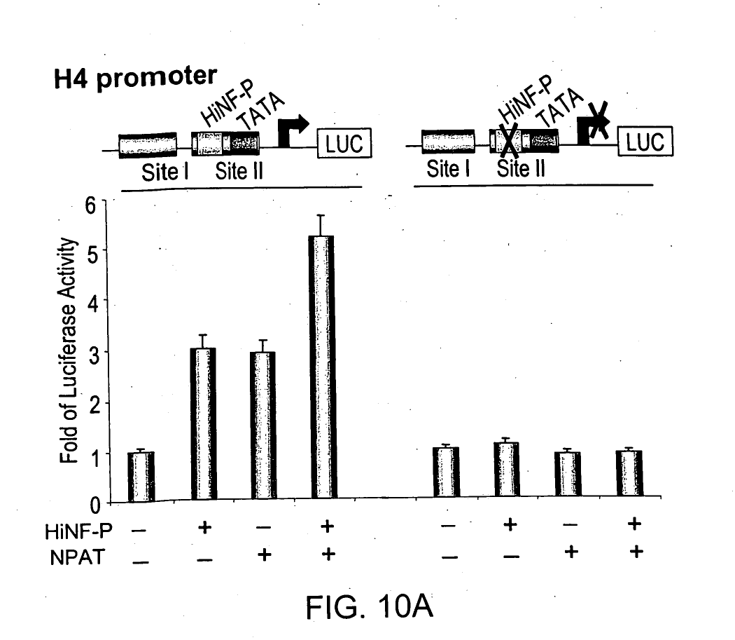

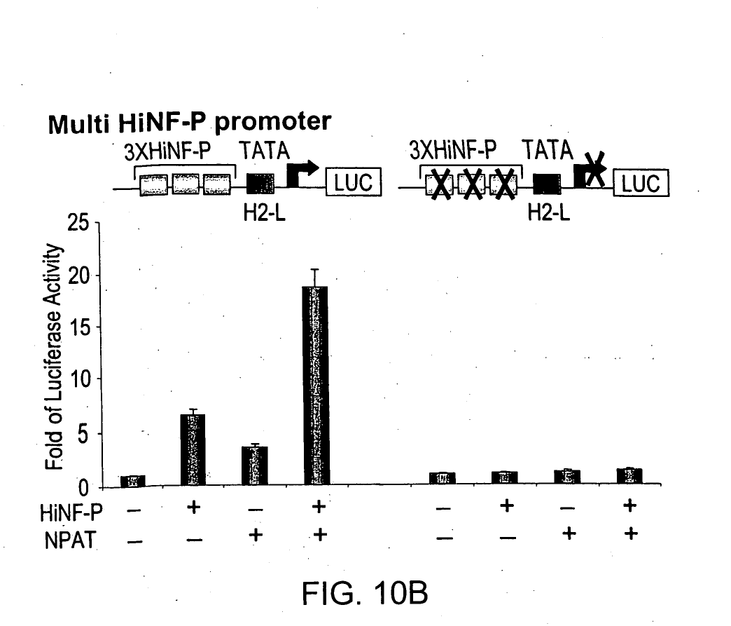

To investigate whether HiNF-P and NPAT-dependent activation of histone H4 gene transcription requires an intact HiNF-P binding element, SAOS-s2 cells were transfected with wild type H4 promoter luciferase reporter construct or a corresponding construct containing mutated HiNF-P binding sites (diagrams are shown at the top of FIG. 10A). Briefly, SAOS2 cells were plated in McCoy's medium supplemented with 15% FBS, 2 mM L-glutamine, 20 unit/ml penicillin, and 20 μg/ml streptomycin at a density of 0.2×10⁶ cells/well of a 6-well plate and incubated overnight at 37° C. Transcription assays were performed using 100 μg of an H4 promoter-luciferase construct or the matching HiNF-P binding site mutant and up to 500 ng of a CMV-driven HiNF-P and/or NPAT expression construct. A total of 0.6 μg plasmid DNA in serum-free medium was mixed with FuGENE-6 (Roche Molecular Biochemical) and incubated with cells for three hours at 37° C. Cells were then supplemented with complete medium and incubated for 36 hours. The culture medium was aspirated and cells were harvested by the addition of 0.2 ml of 1× passive lysis buffer and incubation at room temperature for 10 minutes. Extracts were collected and centrifuged at 10,000 g for 10 minutes at 4° C. Supernatants were used for measurement of luciferase reporter activity. Cell lysates were prepared from 1×10⁷ cells that were harvested 36 hours after transfection with 0.5 μg HiNF-P expression plasmid and assayed by Western blot analysis.

Expression of HiNF-P resulted in an enhancement of histone gene transcription (FIG. 10A). Transient expression of NPAT alone also resulted in activation of histone H4 transcription from the wild type Site II (FIG. 10A). Histone H4 transcription was not increased in the presence of mutant Site II promoter (FIG. 10A), thus demonstrating the specificity of the effect and the requirement for Site II for NPAT activity. Co-expression of HiNF-P and NPAT elevated wild-type promoter activity above that of either factor alone and was greater than additive in magnitude. This demonstrates not only that binding is to Site II, but also that the relationship between HiNF-P and NPAT is synergistic.

Thus, in some embodiments of the invention, both NPAT and HiNF-P expression or activity are increased to increase passage from G1 to S phase and to increase cellular proliferation. Conversely, decreasing expression or activity of one or both of HiNF-P and NPAT can be used as a method of decreasing passage from G1 to S phase and for decreasing cellular proliferation; decreasing expression or activity of both may result in a larger decrease, as the two function synergistically. Furthermore, an NPAT mutant in which CDK2 phosphorylation sites are eliminated cannot activate and inhibit HiNF-P-dependent activation of H4 gene transcription. This demonstrates the requirement for CDK2 phosphorylation sites.

The function of HiNF-P on a chimeric reporter gene containing three copies of the conserved Site II cell cycle regulatory sequences fused to a minimal TATA-box containing promoter was examined. Using this construct, HiNF-P mediated a 6 to 7 fold activation of transcription (FIG. 10B). The corresponding construct in which the three HiNF-P sites are mutated was not responsive to HiNF-P expression. Thus, HiNF-P is a potent activator of histone H4 gene transcription. Not wishing to be bound by theory, the H4 subtype-specific consensus sequence may represent an autonomous element that mediates HiNF-P dependent transcriptional activation of cell cycle dependent H4 genes.

These data indicate that regulation of HiNF-P expression or activity can be an effective means of modulating histone H4 expression and thereby modulate cell proliferation.

To investigate whether NPAT and HiNF-P cooperate to regulate H4 promoter activity, both proteins were expressed together and reporter gene expression was monitored. These experiments are also discussed in FIG. 8 and show that co-expression of NPAT and HiNF-P increased transcription more than expression of either factor alone (FIG. 10A).

Co-expression of CDK2/cyclin E kinase further enhances the transcription of H4. Coexpression of CDK2 inhibitor (p57) reduces transcription of H4. These findings demonstrate that CDK2/cyclin E signaling is important for HiNF-P activity.

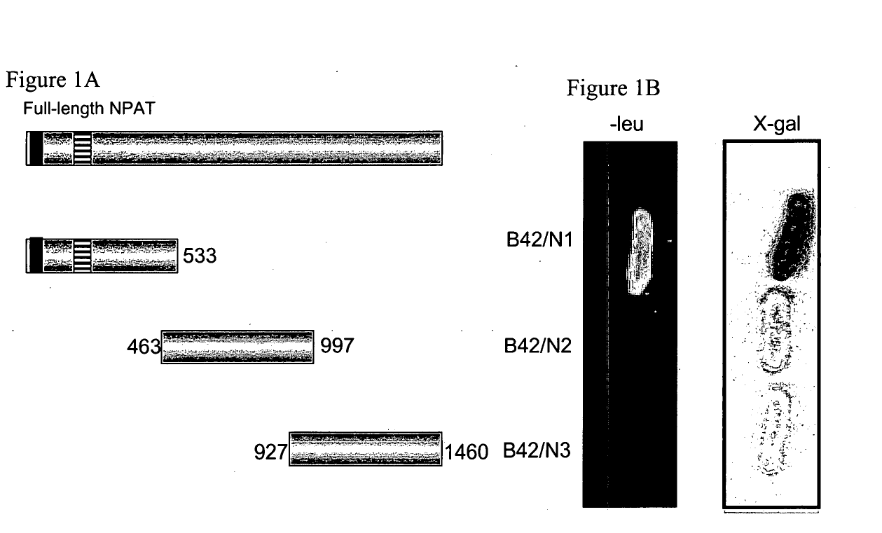

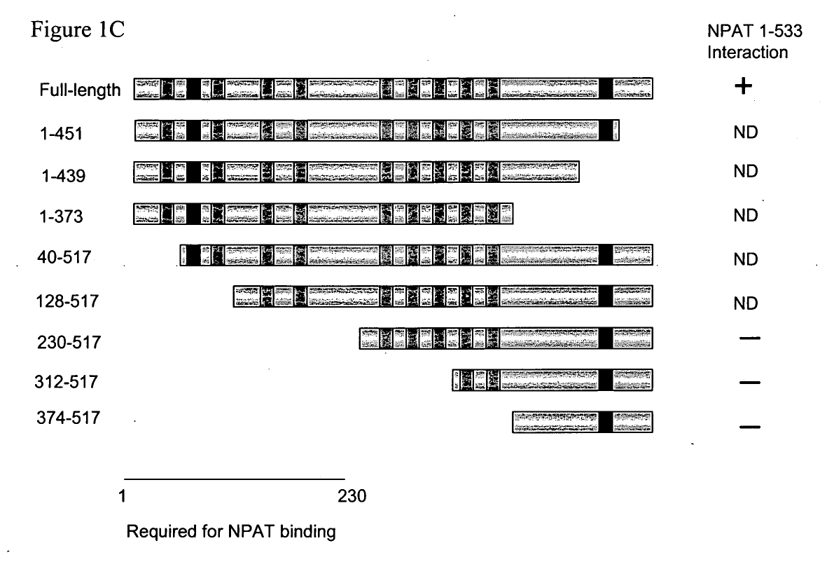

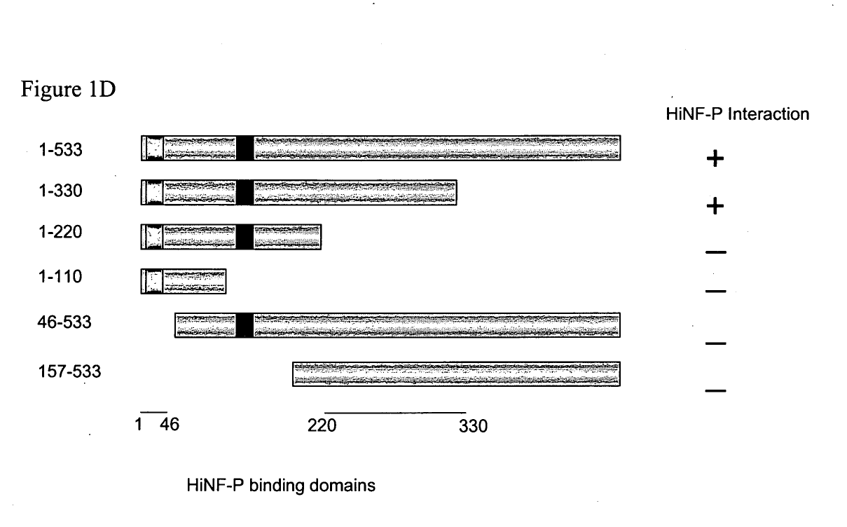

To investigate whether the functional cooperativity between HiNF-P and NPAT reflects a structural linkage in vivo, the LexA/B42 yeast two hybrid system was used. Yeast strain EGY48 according to Aubusel et al. (1997) was used with a panel of expression constructs illustrated in FIG. 1B. These construct express a LexA (DNA binding domain)/HiNF-P fusion protein or fusion proteins containing the B42 transactivation domain fused to segments of NPAT, (B42/N1, B42/N2, and B42/N3). Yeast colonies were analyzed for their activation of the LEU2 and LacZ reporter genes as indicated by growth on leucine deficient media and by a blue color on media containing X-gal. Our data show that the N-terminus of NPAT interacts with HiNF-P (FIG. 1B). The N-terminus of NPAT contains the phylogenetically conserved Lis H domain (black boxes, FIG. 1A) and a segment shown to be necessary for histone transactivation (horizintal stripes, FIG. 1A) (Wei et al., 2003).

To determine which domains of HiNF-P interact with NPAT, deletion constructs of LexA/HiNF-P were generated (shown in FIG. 1C) and co-transformed with B42/N1 into the yeast strain EGY48. Zinc fingers of HiNF-P are represented by gray bars and two glutamic acid rich regions are represented by black bars. To determine which domain of the N-terminal region of NPAT interacts with HiNF-P, further deletion constructs of NPAT were generated (shown in FIG. 1D) and co-transformed with full-length LexA/HiNF-P and assayed for reporter activity. The data show that the regions between amino acids 220 and 330 as well as between amino acids 1 to 46 which contain the Lis H domain are necessary for this interaction

The results demonstrate that HiNF-P and the N-terminal region of NPAT physically interact, and co-expression of HiNF-P and NPAT mutants reveals that the Lis H domain and a second element within the N-terminal region of NPAT are necessary for activation of histone H4 transcription and cooperation with HiNF-P. Chromatin immunoprecipitations reveal that NPAT associates with the H4 locus and antisense mediated depletion of endogenous HiNF-P eliminates this association.

These results demonstrate that NPAT operates via HiNF-P and functions as a co-activator of H4 gene transcription. The finding that multimerized Site II enhances H4 transcription levels is novel and demonstrates a method of increasing levels of H4 transcription by increasing the number of Site II sequences positioned for interaction with H4. Multimerized Site II can also be used to increase transcription of a heterologous sequence (i.e., a sequence that does not encode H4 or is a hybrid sequence comprising some H4 sequence and sequence that is not an H4 sequence). Furthermore, HiNF-P associates with NPAT at Site II to upregulate histone H4 gene expression at the G1/S phase transition and directly links the NPAT/cyclin E/CDK2 signaling pathway to histone gene expression through HiNF-P.

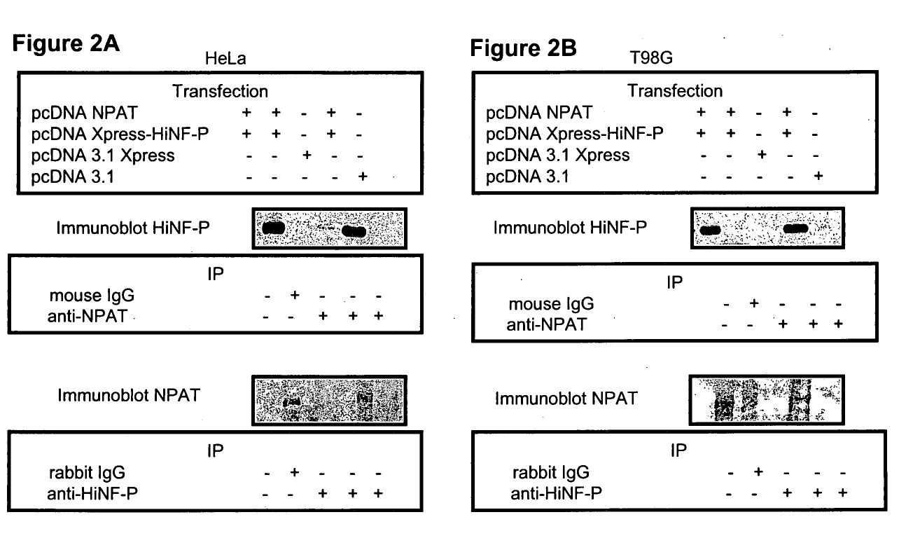

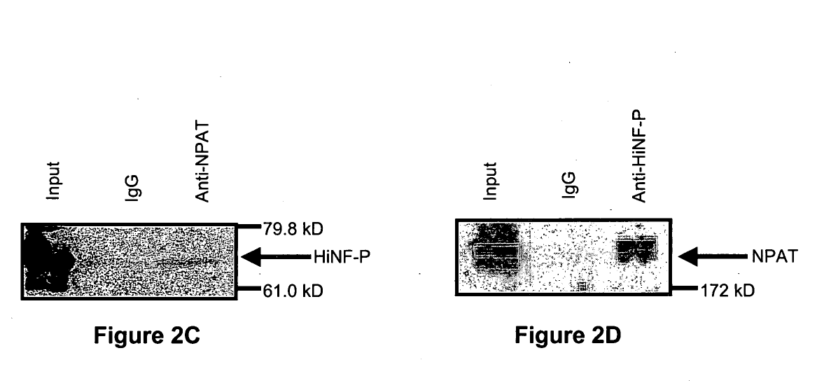

To determine whether NPAT and HiNF-P interact when overexpressed in human carcinoma cell lines, a co-immunoprecipitation experiment was carried out following standard methods. Normal mouse or rabbit IgG was used as a control (lane 2) and immunoprecipitation was carried out, with the indicated antibodies either the mouse monoclonal anti-NPAT or the rabbit polyclonal anti-HiNF-P, as described in the materials and methods. The membranes were probed with either rabbit anti-HiNF-P or mouse anti-NPAT. Referring to FIGS. 2A and B, Lane 1 is 30 ml of cell extract. In lanes 2 and 4 both pcDNA NPAT and pcDNA Xpress-HiNF-P were transfected into both cell lines. Control transfections of pcDNA-Xpress and pcDNA 3.1 appear in lanes 3 and 5 respectively. Normal mouse or rabbit IgG was used as a control (lane 2) and immunoprecipitation was carried out, with the indicated antibodies either the mouse monoclonal anti-NPAT or the rabbit polyclonal anti-HiNF-P, as described in the materials and methods. The membranes were probed with either rabbit anti-HiNF-P or mouse anti-NPAT. The results demonstrate that NPAT and HiNF-P co-immunoprecipitate in both Hela (2A) and T98G (2B) cell lines.

The interactions between endogenous NPAT and HiNF-P were also evaluated in HeLa cells. First, the interaction was evaluated using 400 mg of Hela cell lysate which was subjected to immunoprecipitation with the anti-NPAT antibody (FIG. 2C, third lane). The first lane of FIG. 2C is 40 mg of HeLa cell lysate representing 10% of input and the second lane is the mouse IgG control. The membrane was immunoblotted with chicken anti-HiNF-P.

The reverse pull-down, which examines the interaction of HiNF-P and NPAT by immunoprecipitation, was performed using an anti-HiNF-P antibody with 400 mg of Hela cell lysate (FIG. 2D, third lane). The first lane is 40 mg of HeLa cell lysate representing 10% of input and the second lane is the mouse IgG control. The membrane was immunoblotted with mouse anti-NPAT.

These results demonstrate that endogenous HiNF-P and NPAT interact in vivo in HeLa Cells.

The next question to be answered was whether HiNF-P and NPAT interact directly with each other. Thus, in vitro transcribed and translated (IVTT) NPAT and HiNF-P were mixed at a ratio of 3:1 respectively and immunoprecipitated as described in the material and methods section. The results are shown in FIG. 3. Lane 1 represents the input used for the immunoprecipitations. Lane 2 is a control using non-reactive mouse IgG and Lane 3 is reticulocyte lysate with empty vector pcDNA 3.1 immunoprecipitated with mouse monoclonal anti-NPAT. Lane 4 shows that IVTT HiNF-P will co-immunoprecipitate with IVTT NPAT and this indicates that this interaction does not require a bridging protein. Lane 5 is an immunoprecipitation of the NPAT/HiNF-P mixture using a chicken anti-HiNF-P(IgY) as a control.

### Example 9

### HiNF-P is a Phosphoprotein Based on Metabolic Labeling with Radioactive Phosphate

The phosphorylation status of HiNF-P was evaluated. HeLa cells were transfected with FLAG epitope-tagged HiNF-P expression vector and incubated for 20 hours. The transfected cells were then labeled in vivo with [H₃³²PO₄] for 4 hours. HiNF-P protein was isolated by immunoprecipitation with anti-FLAG monoclonal antibody M2.

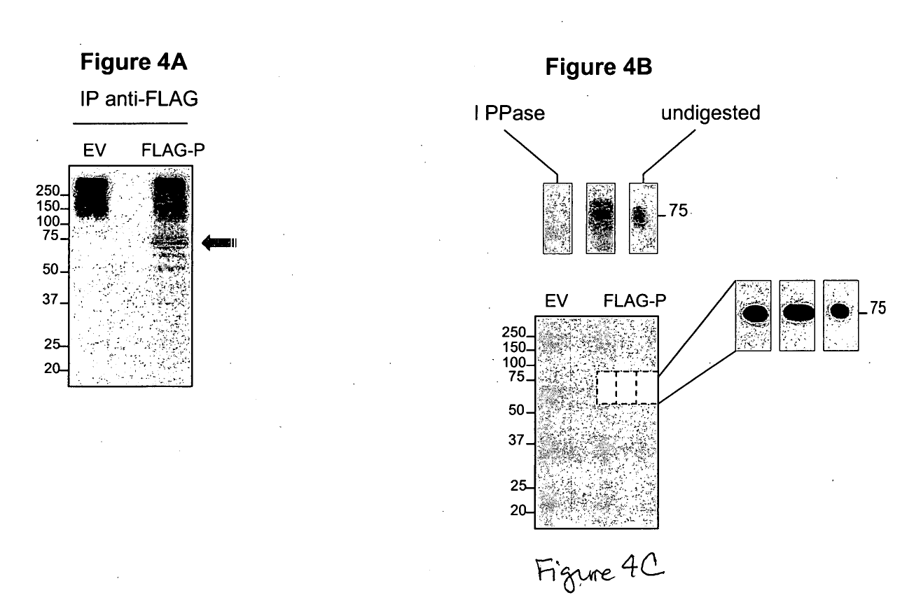

Immunoprecipitates were separated by SDS-PAGE, electrotransferred to a PVDF membrane, and visualized by autoradiography. The band containing ³²P-labeled HiNF-P was excised from the membrane and digested with 1 phosphatase. FIGS. 4A-C show that HiNF-P is phosphorylated (4A) in vivo (arrow), and that this phosphorylation is specific (4B) since the signal was completely removed after 1 phosphatase (1 Ppase) treatment, even though the protein is still present. These results indicate that HiNF-P is a phosphoprotein.

### Example 10

### Antisense Inhibition of HiNF-P Activity Alters Cell Cycle Progression

In some embodiments of the invention, HiNF-P expression is decreased to decrease cell proliferation. A decrease in cell proliferation is useful, e.g., for treating disorders associated with cell proliferation such as cancer. One method of decreasing HiNF-P expression is by introducing into a proliferating cell an antisense oligonucleotide that is targeted to a HiNF-P sequence. The following experiments demonstrate that antisense methods are effective for decreasing proliferation.

In these experiments, antisense oligonucleotides (with the corresponding negative controls; i.e., scrambled and reverse sequences) were tested for their ability to inhibit HiNF-P mRNA levels. Briefly, SAOS2 cells were cultured and transfected when approximately 60-70% confluent. Cells were washed twice with PBS and transfected with each oligonucleotide (Oligo Etc., Inc.) at 400 nM with Lipofectin™ (Invitrogen), following the manufacturer's instructions. The oligonucleotides used were: 5′-GGG CAT TGG TCT GAT TCA CC-3′ SEQ ID NO:17 (antisense), 5′-CCA CTT AGT CTG GTT ACG GG-3′ SEQ ID NO:18 (reverse), and 5′-AGG CGT TGA TCT CAT TAA CC-3′ SEQ ID NO:19 (scramble). Oligonucleotide and Lipofectin™ were separately diluted in 500 μl of Opti-MEM (Gibco). After incubation at room temperature for 45 minutes the solutions were mixed and incubated for an additional 15 minutes. The Lipofectin™/oligonucleotide mixture was supplemented with Opti-MEM medium to a final volume of 5 ml and then added dropwise to the cells. After a four hour incubation at 37° C., the cells were supplemented with 5 ml of complete medium containing 30% FBS. After incubation for 30 hours, the cells were then assayed for inhibition of target gene expression and cell cycle distribution.

Cells treated with the HiNF-P antisense oligonucleotide that was directed against a segment in the 3′ end of the HiNF-P mRNA) exhibited a greater than 90% reduction in HiNF-P mRNA and protein levels, whereas the related scrambled and reverse oligonucleotides did not influence the endogenous mRNA levels of HiNF-P.

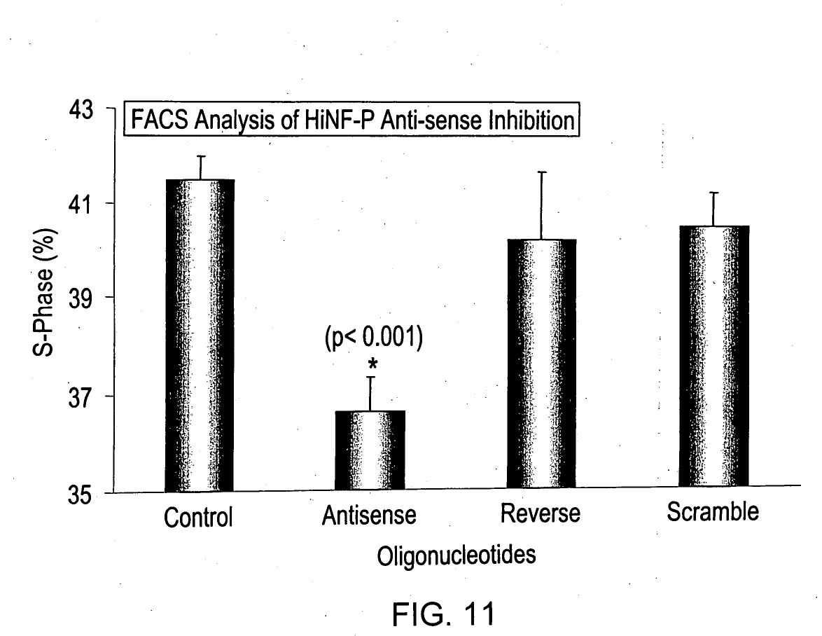

Actively proliferating cells were incubated with this HiNF-P antisense oligonucleotide, scrambled oligonucleotide, reverse oligonucleotide, and control (no oligonucleotide), and cell cycle distribution was monitored using flow cytometry (FIG. 11). The reduction of cells in S phase in samples treated with antisense oligonucleotide (average value 36%) relative to control cells (average value 42%) was consistently observed in all experiments. The antisense-induced reduction in HiNF-P levels thus resulted in a quantitatively statistically significant 10-15% reduction in the fraction of cells progressing through S phase. In contrast, scrambled and reverse oligonucleotides did not affect cell cycle distribution.

Western and Northern blot analysis revealed that HiNF-P levels were downregulated by at least one order of magnitude.

These data demonstrate that HiNF-P activity is required for efficient cell cycle progression in proliferating cells. Furthermore, the data show that inhibiting HiNF-P expression (e.g., by introducing antisense oligonucleotides into a cell) is an effective method of reducing cellular proliferation.

### Example 11

### HiNF-P is a Cell-Context Dependent and p57/Kip2 Sensitive Bi-Functional Regulator of Histone H4 Gene Transcription at the G1/S Phase Transition

Transcription factor HiNF-P is a critical link in a growth factor dependent signaling pathway that uses NPAT, a Cyclin E/CDK2 substrate, as a co-activator to modulate H4 promoter activity. Histone genes, which encode the essential constituents of chromatin that are required to package newly replicated DNA, are by necessity transcribed in actively proliferating normal and tumor cells.

The potent activating properties of HiNF-P are evident in many cell lines (e.g., SAOS-2 osteosarcoma cells), but HiNF-P functions as a modest repressor in other cell types (e.g., HeLa S3 cervical carcinoma cells). Forced expression of HiNF-P in HeLa S3 cells downregulates H4 promoter activity, while NPAT is transcriptionally neutral. Thus, a bypass or inactivation of signals mediated by the CDK2/Cyclin E/NPAT pathway may switch the transcriptional functionality of HiNF-P.

Because the CDK inhibitory protein p57/Kip2 can block CDK2/Cyclin E activity, we tested whether differences in p57 levels influence HiNF-P activity. When HiNF-P and NPAT function as stimulatory proteins (e.g., in SAOS-2), levels of p57 are very low or undetectable. In contrast, when p57 is present at high levels, HiNF-P acts as a repressor and NPAT is transcriptionally inert (e.g., in HeLa S3). Forced expression of p57 in SAOS-2 cells inhibits activation of H4 transcription by endogenous HiNF-P, but not when the HiNF-P element is mutated. Similarly, p57 blocks HiNF-P/NPAT co-activation and an NPAT mutant lacking CDK2 phosphorylation sites acts as a dominant-negative inhibitor of HiNF-P activity.

Hence, HiNF-P functions as a bi-functional regulator that modulates H4 gene transcription at the G1/S transition, depending on the kinase status of the CDK2/Cyclin E/NPAT pathway.

### Example 12

### HiNF-P Knockout Mice

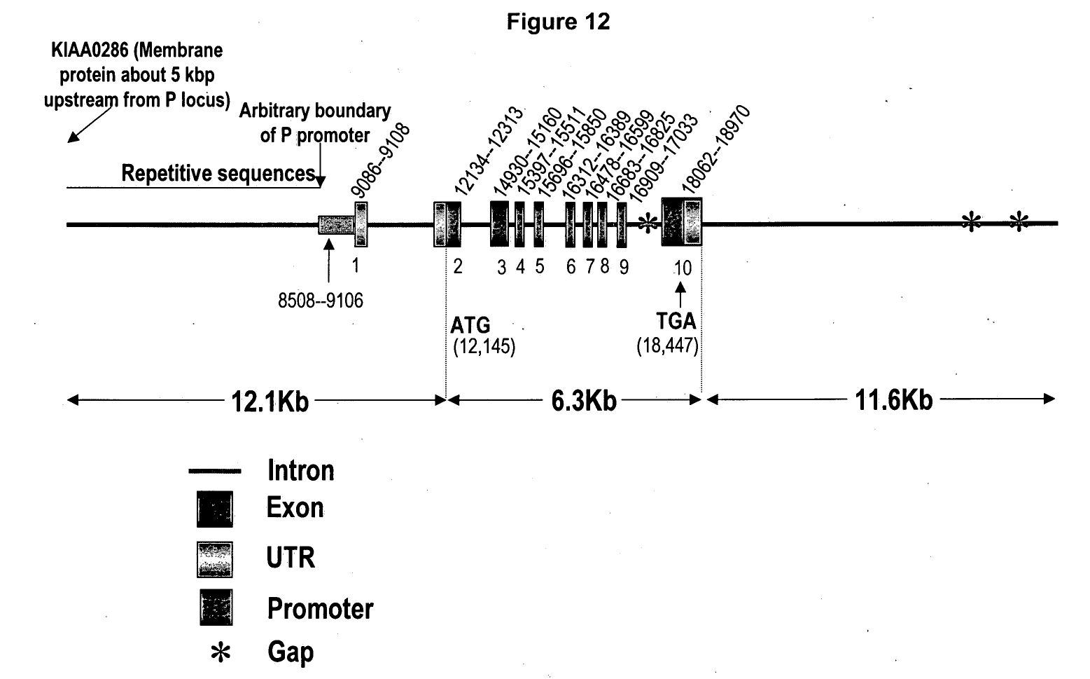

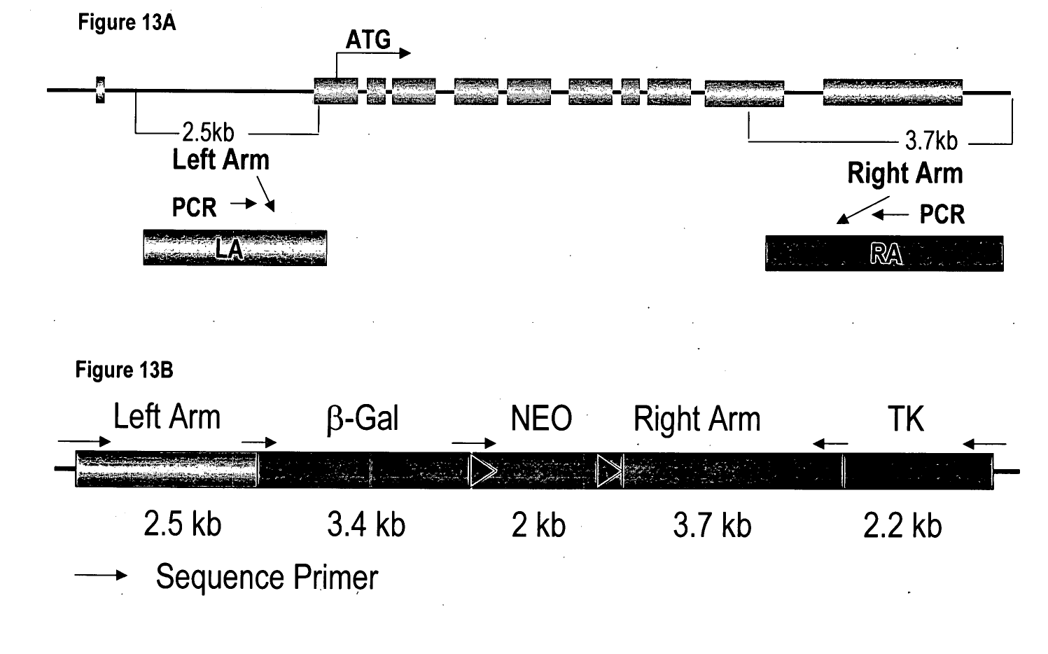

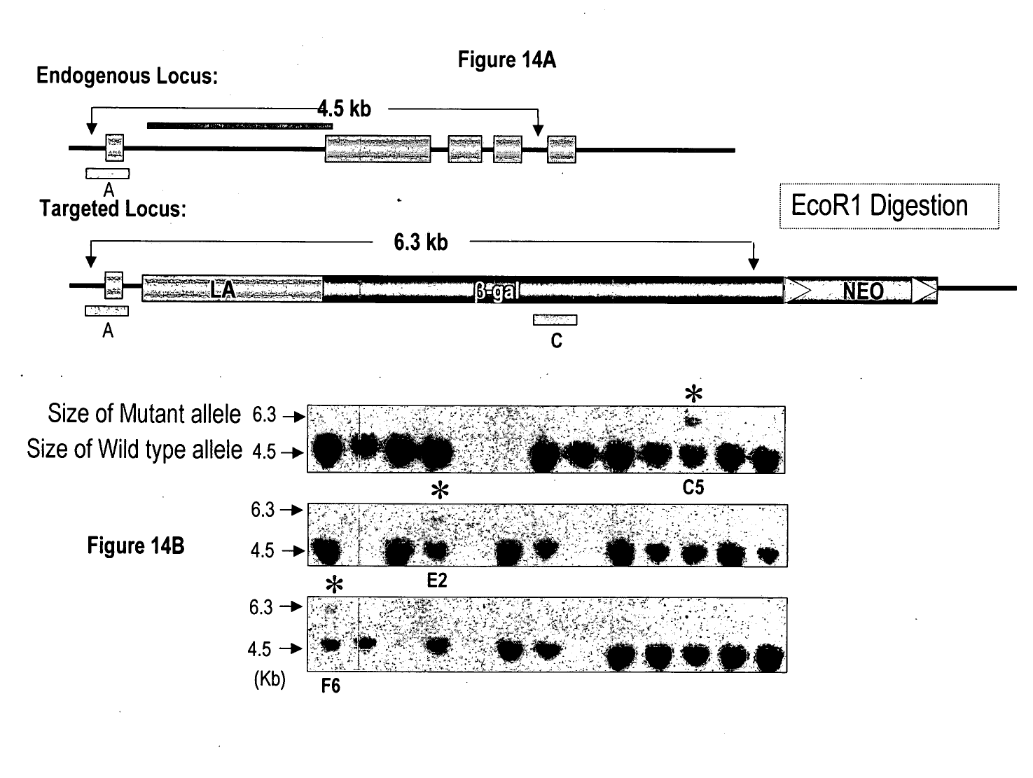

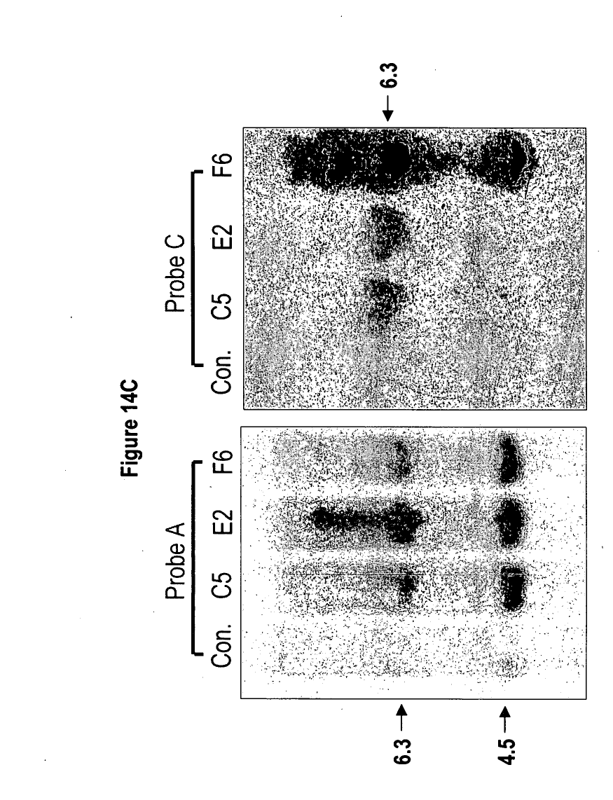

Transgenic mice have been generated using a targeting vector that ablates one allele of the HiNF-P gene. A diagram of the genomic structure of the mouse HiNF-P gene is shown in FIG. 12. A HiNF-P knock out construct targeting vector was constructed as illustrated in FIGS. 13A and B. FIG. 13A is a diagram showing the regions of the genomic sequence that were used for the left and right arms of the construct (which is illustrated in FIG. 13B). This vector was incorporated the construct into mouse embryonic stem (ES) cells by homologous recombination. The mice were generated using standard methods. Briefly, the ES cells were injected into blastocysts and germline transmission in the mouse has been achieved. FIG. 14A shows the endogenous and targeted HiNF-P loci, illustrating the expected sizes after EcoR I digestion (4.5 and 6.3 kb, respectively), and the placement of probes A and C. FIG. 14B is a representation of an autoradiogram showing the results of digestion of a number of ES cell clones with Eco RI. Each lane contains DNA from a different clone. Positive ES clones C2, E5, and F6, as identified by Southern blot analysis, are indicated with an asterisk. FIG. 14C shows the results of Southern blot analysis, confirming that each of the three distinct ES clones (C2, E5, and F6) each contain only one targeted HiNF-P allele.

At present, the heterozygous HiNF-P null mice are being bred to homozygosity. Tail biopsies will be used to extract DNA, to confirm the genotype of the mice by standard Southern blotting and PCR-based methodology, and for analysis of β-gal expression by Western blotting.

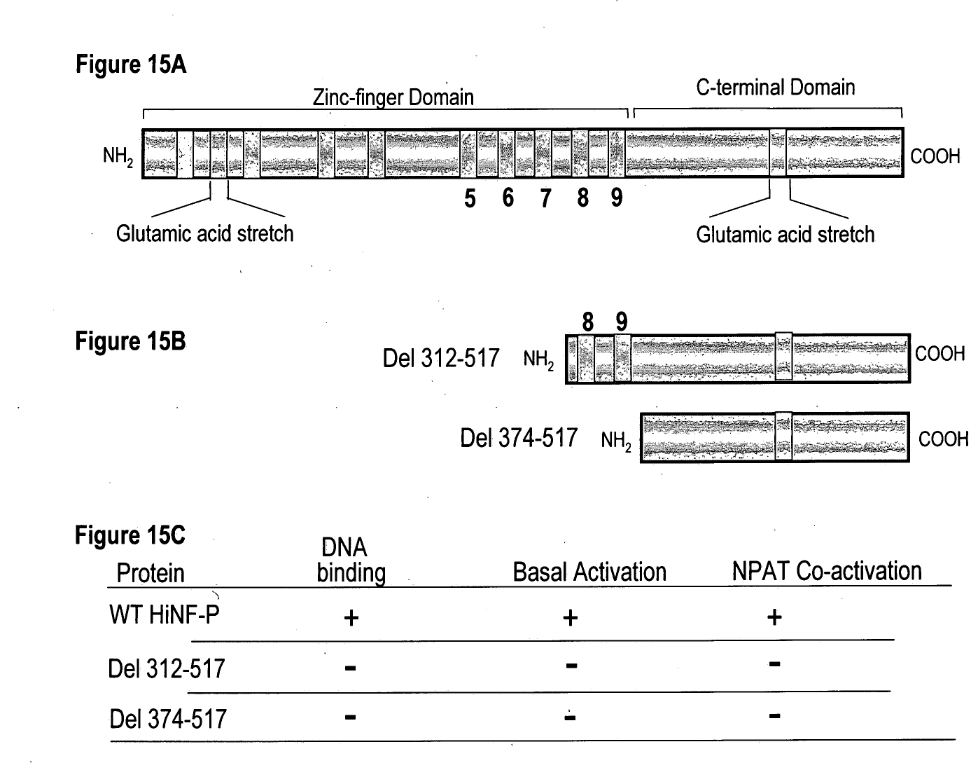

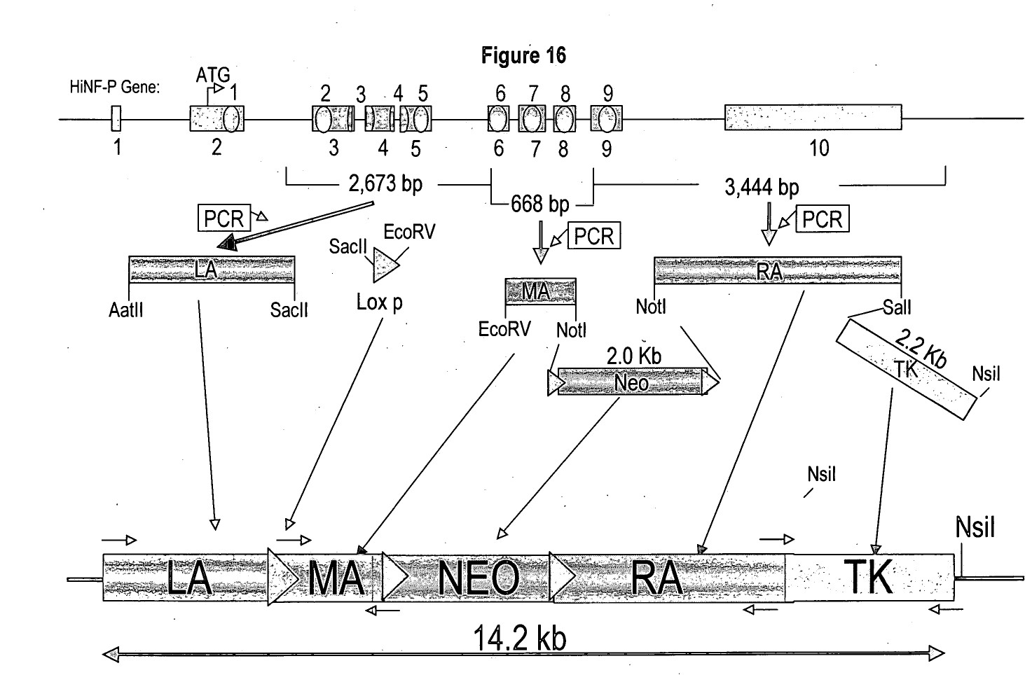

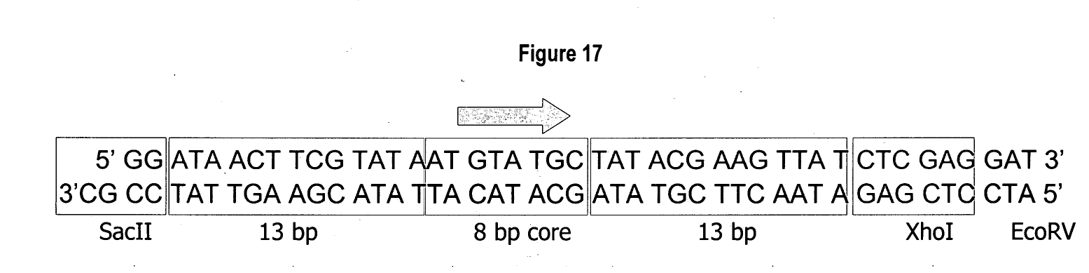

Because the HiNF-P homozygous knock-out mouse may exhibit very early embryonic lethality, a novel targeting vector that permits conditional ablation of the HiNF-P locus has also been prepared using Cre-Lox technology to direct site-specific recombination (see Orban et al., Proc. Natl. Acad. Sci. USA 89(15):6861-5 (1992); Akagi et al., Nuc. Acids Res. 25(9):1766-1772 (1997); Lakso et al., Proc. Natl. Acad. Sci. USA 89:6232-6236 (1992); Rossant and McMahon, Genes Dev. 13(2)142-145 (1999); Wang et al., Proc. Natl. Acad. Sci. USA 93:3932-3936 (1996)). FIG. 15A illustrates the HiNF-P protein domain structure, illustrating the nine Zinc-finger domains, glutamic acid-rich regions, and the C-terminal domain. Two additional HiNF-P deletion mutants, Del 312-517 and Del 374-517, were constructed (FIG. 15B); the activities of the HiNF-P wild type and deletion mutants are illustrated in FIG. 15C. FIG. 16 illustrates the conditional HiNF-P knock out targeting vector, including a floxed neomycin resistance gene (Neo) (flanked by lox sequences, illustrated by triangles). RA, right arm, MA, middle arm, LA, left arm.

### Example 13

### Coupling of HiNF-P, Histone H4 Gene Expression, and Cell Proliferation

The EMSA and Western blot methods illustrated in Example 7 above can also be used to demonstrate HiNF-P expression and activity, and in screens for compounds that modulate HiNF-P expression or activity. A test compound is incubated with proliferating HL-60 cells, and the ability of HiNF-P to bind to DNA in the presence and absence of the test compound is assayed. Western blot analysis and EMSA is then used to determine whether the amount of HiNF-P is affected by the presence of the test compound.

### OTHER EMBODIMENTS

A number of embodiments of the invention have been described. Nevertheless, it will be understood that various modifications can be made without departing from the spirit and scope of the invention. Accordingly, other embodiments are within the scope of the following claims.

What is claimed is:

1. A method of enhancing cellular proliferation, the method comprising introducing into a cell a compound that alters the expression or activity of a Histone Nuclear Factor P (HiNF-P) polypeptide, in an amount effective to enhance proliferation of the cell.

2. The method of claim 1, wherein the compound is a nucleic acid molecule comprising a sequence encoding a HiNF-P polypeptide, which is introduced into the cell under conditions such that the HiNF-P polypeptide is expressed.

3. The method of claim 2, wherein the nucleic acid molecule encoding the HiNF-P polypeptide is in an expression vector.

4. The method of claim 1, wherein the compound is an HiNF-P polypeptide.

5. The method of claim 1, wherein the compound increases the number of HiNF-P polypeptides.

6. The method of claim 5, wherein the activity that is altered results in enhanced expression of a histone H4 gene.

7. The method of claim 1, wherein the cell is a cultured cell.

8. The method of claim 1, wherein the cell is in a living mammal.

9. The method of claim 8, wherein the living mammal is a human.

10. The method of claim 1, further comprising introducing into the cell a nucleic acid molecule encoding an Nuclear Protein, Ataxia-Telangiectasia locus (NPAT) polypeptide, under conditions such that the NPAT polypeptide is expressed.

11. The method of claim 10, wherein the nucleic acid molecule encoding the NPAT polypeptide is in an expression vector.

12. The method of claim 8, wherein the mammal has a condition that would benefit from increased cell proliferation.

13. The method of claim 12, wherein the condition is a wound, and the compound enhances healing of the wound.

14. A method of increasing transcription of a pre-selected target nucleic acid sequence in a cell, the method comprising:
introducing an expression vector into a cell, the vector comprising a Histone Nuclear Factor P (HiNF-P) binding site operatively linked to the target sequence and a regulatory element effective to enable transcription of the target nucleic acid sequence in the cell when a HiNF-P polypeptide is expressed in the cell; and
culturing the cell or progeny of the cell under conditions that enable expression of the HiNF-P polypeptide, thereby increasing transcription of the target nucleic acid sequence.

15. The method of claim 14, wherein the HiNF-P binding site comprises a Site II promoter sequence.

16. The method of claim 14, wherein the vector comprises at least two copies of the HiNF-P binding site.

17. The method of claim 14, wherein the transcribed nucleic acid sequence is translated.

18. The method of claim 14, further comprising introducing into the cell an expression vector comprising a nucleic acid sequence encoding a HiNF-P polypeptide, and culturing the cell to express the HiNF-P polypeptide.

19. The method of claim 14, further comprising introducing into the cell an expression vector comprising a nucleic acid sequence encoding a Nuclear Protein, Ataxia-Telangiectasia locus (NPAT) polypeptide, and culturing the cell to express the NPAT polypeptide.

20. An isolated polypeptide comprising a sequence selected from the group consisting of VRYESVELTQQLLRQPQE (SEQ ID NO:3) and MEKLQGIAEE PEIQMV (SEQ ID NO:4).

21. An isolated antibody or portion thereof that specifically binds to a Histone Nuclear Factor P (HiNF-P) polypeptide.

22. The isolated antibody of claim 21, wherein the antibody specifically binds to an amino acid sequence selected from the group consisting of VRYESVELTQQLLRQPQE (SEQ ID NO:3) and MEKLQGLAEEPEIQMV (SEQ ID NO:4).

23. A method of identifying a compound that modulates expression of an H4 histone gene, the method comprising:
obtaining a cell comprising:
(a) a reporter gene operatively linked to a Histone Nuclear Factor P (HiNF-P) binding site, such that when a HiNF-P polypeptide binds to the HiNF-P binding site the reporter gene is expressed, and
(b) a nucleic acid sequence encoding a HiNF-P polypeptide that is expressed;
contacting the cell with a test compound; and
measuring the level of expression of the reporter gene,
wherein a difference in the level of expression of the reporter gene relative to a control cell that was not contacted with the test compound indicates that the test compound modulates the expression of a H4 histone gene.

24. The method of claim 23, wherein the reporter gene is an H4 histone gene.

25. The method of claim 23, wherein the HiNF-P binding site is a Site II promoter sequence.

26. The method of claim 23, wherein expression of the reporter gene is increased relative to the control.

27. The method of claim 23, wherein expression of the reporter gene is decreased relative to the control.

28. The method of claim 23, wherein the test compound is selected from the group consisting of an antisense oligonucleotide and an siRNA.

29. The method of claim 23, wherein the test compound is a peptidomimetic, peptoid, aptamer, carbohydrate, polysaccharide, non-nucleic acid small organic molecule, inorganic molecule, polypeptide, antibody, or ribozyme.

30. An isolated antisense nucleic acid that inhibits expression of a Histone Nuclear Factor P (HiNF-P) polypeptide.

31. The isolated antisense nucleic acid of claim 30, comprising the sequence 5′-GGCATTGGTCTGATTCACC-3′ (SEQ ID NO:10).

32. A method of decreasing proliferation of a cell, the method comprising:
administering to the cell a composition in an amount sufficient to inhibit Histone Nuclear Factor P (HiNF-P) expression or activity in the cell, thereby decreasing proliferation of the cell.

33. The method of claim 32, wherein the compound administered to the cell is a HiNF-P antisense oligonucleotide.

34. The method of claim 32, wherein the compound administered to the cell is an siRNA that is targeted to a HiNF-P nucleic acid sequence.

35. The method of claim 32, further comprising administering a second composition in an amount sufficient to inhibit Nuclear Protein, Ataxia-Telangiectasia locus (NPAT) expression or activity.

36. A method for detecting a Histone Nuclear Factor P (HiNF-P) polypeptide in a biological sample, the method comprising:
obtaining a biological sample,
contacting the biological sample with an antibody that specifically binds to a HiNF-P polypeptide under conditions that enable the formation of a HiNF-P polypeptide-antibody complex; and
detecting any HiNF-P polypeptide-antibody complexes, wherein the presence of a complex indicates the presence of a HiNF-P polypeptide in the sample.

37. A method of treating a subject having a disorder characterized by excessive cell proliferation, the method comprising administering to the subject a compound that inhibits Histone Nuclear Factor P (HiNF-P) expression or activity, in an amount effective to decrease cell proliferation.

38. The method of claim 37, wherein the disorder is cancer.

39. The method of claim 37, wherein the compound administered to the subject is a HiNF-P antisense oligonucleotide.

40. The method of claim 37, wherein the compound administered to the subject is an siRNA that is targeted to a HiNF-P nucleic acid sequence.

41. A transgenic non-human mammal whose somatic and germ cells comprise a disrupted Histone Nuclear Factor P (HiNF-P) allele, wherein the cells, if the mouse is homozygous, exhibit decreased HiNF-P activity as compared to a wildtype mouse.

42. The transgenic non-human mammal of claim 41, wherein the mammal is a mouse.

43. The transgenic mouse of claim 42, whose cells comprise two disrupted HiNF-P alleles.

44. The transgenic mouse of claim 42, whose cells additionally comprise a disrupted Nuclear Protein, Ataxia-Telangiectasia locus (NPAT) allele.

45. A transgenic mouse whose cells comprise a conditional transgene that modulates the expression of a Histone Nuclear Factor P (HiNF-P) polypeptide, wherein under pre-selected conditions the transgene increases or decreases the expression of the HiNF-P polypeptide.

46. The transgenic mouse of claim 45, wherein the conditional transgene decreases the expression of the HiNF-P polypeptide.

47. The transgenic mouse of claim 45, wherein the conditional transgene increases the expression of the HiNF-P polypeptide.

48. A targeting vector comprising a nucleic acid sequence that disrupts expression of a Histone Nuclear Factor P (HiNF-P) polypeptide when introduced into a cell, the vector comprising a first and second sequences complementary to first and second regions of a nucleic acid sequence encoding a HiNF-P polypeptide, and a third sequence inserted between the first and second sequences, wherein the third sequence is not complementary to a nucleic acid sequence encoding a HiNF-P polypeptide.

49. The targeting vector of claim 48, wherein the third sequence is a reporter gene.

50. The targeting vector of claim 49, wherein the reporter gene encodes β-galactosidase.

51. The targeting vector of claim 48, wherein the nucleic acid sequence disrupts expression of a HiNF-P polypeptide by homologous recombination into an endogenous HiNF-P allele.

52. The targeting vector of claim 48, further comprising a regulatory sequence capable of conditionally disrupting expression of a HiNF-P allele.

53. The targeting vector of claim 52, wherein the regulatory sequence is a Lox sequence.
